(19) United States
(12) Patent Application Publication
(10) Pub. No.: US 2015/0119262 A1
(43) Pub. Date: Apr. 30, 2015

(54) PREDICTION OF GENE TARGETS OF ENHANCERS

(71) Applicant: Case Western Reserve University, Cleveland, OH (US)

(72) Inventor: Peter C/ Scacheri, Cleveland, OH (US)

(21) Appl. No.: 14/524,853

(22) Filed: Oct. 27, 2014

Related U.S. Application Data
(60) Provisional application No. 61/895,684, filed on Oct. 25, 2013.
(60) Provisional application No. 61/897,540, filed on Oct. 30, 2013.

(51) Int. Cl. G06F 19/22; C12Q 1/68

(52) U.S. Cl. CPC G06F 19/22; C12Q 1/6883; C12Q 2600/154; C12Q 2600/158
USPC 506/8; 506/39; 702/19

(57) ABSTRACT

This disclosure relates generally to the prediction of a gene target of an enhancer based on genomic proximity and a comparison to a set of known enhancer-gene pairs. A gene can be selected a gene from a given cell type. A domain can be established bi-directionally around the gene, and enhancers located within the domain can be identified. The enhancers located within the domain can be considered to be in close genomic proximity to the gene. For each enhancer, a test pair of the enhancer and the gene can be established and compared to a set of known enhancer-gene pairs. The prediction can be established based on the comparison and the genomic proximity.

### CROSS-REFERENCE TO RELATED APPLICATIONS

This application claims the benefit of U.S. Provisional Patent Application No. 61/895,684, filed Oct. 25, 2013, entitled ENHANCER-GENE CORRELATION, and U.S. Provisional Patent Application No. 61/897,540, filed Oct. 30, 2013, entitled ENHANCER-GENE CORRELATION. The subject matter of these applications is incorporated herein by reference in their entirety.

### FIELD OF THE INVENTION

This disclosure relates generally to the prediction of gene targets of enhancers based on genomic proximity and a comparison to a set of known enhancer-gene pairs.

### BACKGROUND

Many single nucleotide polymorphisms (SNPs) that predispose to common diseases are “enhancers,” deoxyribonucleic acid (DNA) sequences that fall outside protein coding genes in noncoding regulatory elements, but can control the efficiency and the rate of transcription of a gene. Although thousands of SNPs have been mapped to regulatory elements, more than 80% of the associated target genes are unknown. The assignment of enhancers to their target genes is complex because, although enhancers are usually cis-acting, the enhancer can be located upstream or downstream from its target gene at a large distance away from the target gene. Additionally, a single enhancer can influence the expression of multiple genes.

Enhancer-gene interactions have been predicted utilizing different methods, but these methods suffer from drawbacks. The most common predictor of enhancer-gene interactions includes, assigning an enhancer to the nearest gene. However, this only serves as an approximation of enhancer-gene interactions since enhancers need not interact with genes that are nearest in location. Experimental methods to delineate genome interactions include Chromatin Conformation Capture (3C), Circular 3C (4C), 3C Carbon Copy (5C), genome wide 3C (Hi-C) and Chromatin Interaction Analysis by Paired-End Tag sequencing (ChIA-PET), collectively referred to as “C” methods. These “C” methods provide valuable information about higher-order chromatin structure and genome organization. However, they are laborious and only half of all detected interactions are concordant among replicate experiments, and these methods may not validate with independent non-“C” based approaches, such as DNA fluorescence in-situ hybridization (FISH). Computational approaches for connecting enhancers to genes use comparative analyses of chromatin features, such as DNase I hypersensitive sites (DHSs) and histone modifications identified across multiple cell types, to elucidate enhancer-gene interactions in the genome. These computational approaches suffer accuracy and efficacy deficiencies. Consequently, the impact of enhancer SNPs on target transcript levels, cell function and disease risk is still unknown.

### SUMMARY

This disclosure describes systems and methods that can employ a computational approach that can facilitate the prediction of gene targets of enhancers. Unlike previous solutions, the computational approach employed by the systems and methods described herein can provide accurate information in an efficient manner. The information can be based on genomic proximity between the enhancer and the gene and a comparison to known enhancer-gene pairs.

In accordance with an aspect of this disclosure, a system is provided that can provide information that can be used to predict a gene target of an enhancer. The system can include a non-transitory memory that stores machine-readable instructions and a processor configured to access the memory and execute the machine-readable instructions. The machine-readable instructions can include a gene selector that selects a gene from a given cell type. The machine-readable instructions can also include a domain selector that identifies an enhancer located within a domain established across the gene. The machine-readable instructions can also include a correlater that tests a pair of the enhancer and the gene based on a comparison to a set of known enhancer-gene pairs.

In accordance with another aspect of this disclosure, a method is provided that can provide information that can be used to predict a gene target of an enhancer. The method can be performed at a server that includes a non-transitory memory and a processor. The method can include selecting a gene from a given cell type. The method can also include establishing a domain across the gene. The method can also include identifying an enhancer located within the domain. The method can also include comparing a pair of the enhancer and the gene to a known set of enhancer-gene pairs.

In accordance with a further aspect of this disclosure, a non-transitory computer readable storage medium storing machine-readable instructions that when executed by a processor facilitate the performance of operations that can be used to predict a gene target of an enhancer. The operations can include identifying a gene from a given cell type based on an expression level. The operations can also include locating an enhancer within a domain established across the gene. The operations can also include comparing the enhancer and the gene to a set of known enhancer-gene pairs. The operations can also include predicting whether the enhancer and the gene are associated based on the comparison.

### BRIEF DESCRIPTION OF THE DRAWINGS

The features, objects, and advantages of the invention will become more apparent from the detailed description set forth below when taken in conjunction with the drawings, wherein:

FIG. 1 is a schematic block diagram of an example that can provide information that can be utilized to determine whether a gene is a target of an enhancer in accordance with an aspect of the present disclosure;

FIG. 2 illustrates two conditions governing an example of selection of a domain related to a gene that can be used to determine one or more enhancers within genomic proximity of a gene in accordance with an aspect of the present disclosure;

FIG. 3 is a schematic block diagram showing an example comparison between a proposed gene-enhancer pair and a set of known enhancer-gene pairs in accordance with an aspect of the present disclosure;

FIG. 4 is a process flow diagram of an example of a method for determining information that indicates whether a gene is a target of an enhancer in accordance with an aspect of the present disclosure; and

FIG. 5 is a schematic block diagram of an example system of hardware components capable of implementing examples of the systems and methods disclosed in FIGS. 1-4.

FIGS. 6-11 illustrate the PreSTIGE methodology and approximation of a FDR. FIG. 6 shows how PreSTIGE utilizes comparative analysis of H3K4me1 levels and transcript levels across a diverse panel of cell types to elucidate enhancer-gene interactions within a specified domain that is defined by distance and CTCF binding sites. FIG. 7 shows the distribution of interactions based on the number of cell types in which the prediction is made. FIG. 8 is a UCSC browser image of a representative lost enhancer locus (lost VEL). FIG. 9 is an approximation of the FDR (mean+/−SEM) based on colon cancer data for PreSTIGE predictions compared to five commonly used computational methods. FIG. 10 shows the expression fold change (cancer/colon crypt) for genes within the domains of lost enhancers. FIG. 11 is a heatmap showing overall correlation between VELs and gene expression.

FIGS. 12-15 illustrate the evaluation of predictions made by PreSTIGE methodology. FIG. 12 illustrates the number of enhancers involved in low stringency (A) and high stringency (B) predictions. FIG. 13 shows the percent of predicted interactions that involve enhancer and the nearest gene for each cell type. FIG. 14 shows the number of enhancers predicted to regulate each gene. FIG. 15 shows the number of gene targets predicted for each enhancer.

FIGS. 16-18 illustrate that PreSTIGE predictions can reveal targets of disease-associated SNPs that map to enhancers. FIG. 16 illustrates a H3K4me1 signal at the TCF7L2 locus. The SNP re7903146 correlated to Type 2 Diabetes risk is found in HepG2 specific enhancer is predicted to target TCF7L2. FIG. 17 shows a hierarchical clustering of disease traits based on the number of SNPs that intersect with cell type-specific PreSTIGE predictions. FIG. 18 shows a cluster of disease traits that correlate with SNPs present in GM12878 (B-lymphoblast) enhancers.

FIGS. 19-22 illustrate the impact of immune disorder SNPs on transcript levels of highly cell type-specific genes. FIG. 19 illustrates rheumatoid arthritis SNPs (A) and Crohn's disease SNPs (B). FIG. 20 illustrates the expression of transcripts in B-lymphoblast cells from individuals carrying the risk allele compared to those homozygous for the non-risk allele for 6 genes associated with rheumatoid arthritis enhancer-SNPs. FIG. 21 illustrates the percent of H3K4me1 sites that are associated with a PreSTIGE prediction. FIG. 22 illustrates cell type-specificity (Shannon entropy Q score) of all enhancers in the 12-cell type comparator set, GM12878 enhancers, and enhancers containing disease associated SNPs.

FIGS. 23-26 illustrate that common disease SNPs often involve multiple enhancer variants in LD. FIG. 23 shows a percent of enhancer variants that are in LD with SNPs that fall in additional enhancer with the same predicted gene target for, non-disease associated B-cell enhancer SNPs and disease associated B-cell enhancer SNPs (Fischer's exact test P<0.0001). FIG. 24 shows a UCSC browser view of H3K4me1 ChIP-seq data from GM12878 cells. FIG. 25 shows SOCS1 transcript levels in B-lymphoblasts from individuals homozygous for the non-risk SNP, heterozygous and homozygous for the risk SMP. FIG. 26 illustrates the expression of SOCS1 in individuals separated by their halotypes.

FIGS. 27-30 illustrate how enhancer profiles can distinguish mouse pluripotent states. FIG. 27 shows VENN diagrams showing the number and overlap of expressed transcripts, H3K27ac+ promoters of expressed genes, and H3K27ac+ enhancers detected in mESCs and mEpiSCs. FIG. 28 is a heatmap of expression differences between mESCs and mEpiSCs. FIG. 29 shows (A) row normalized expression of transcripts differentially expressed between mESCs and mEpiSCs, (B) illustrates aggregate plots depicting average ChIP-seq and DNase-seq signals of promoters and enhancers of mESC-enriched genes in mESCs and mEpiSCs, and (C) is similar to (B) except for mEpiSC-enriched genes. FIG. 30 shows windowed heatmaps contrasting DNase HS, H3me1, H3K27ac, and H3K27me3 signal =1-5 kb from the midpoiont of DNase-centered putative enhancers identified in mESCs or mEpiSC.

FIGS. 31-38 illustrate how pluripotency-enriched genes show dramatic enhancer differences between pluripotent states. FIG. 31 shows a heatmap depicting row normalized expression of pluripotency-enriched genes. These genes are not differentially expressed, have <2-fold change between mESCs and mEpiSCs, and are relatively specific to mESCs and mEpiSCs compared to downstream tissues. FIG. 32 shows aggregate plots of DNase hypersensitivity and histone marks at the promoters of pluripotency-enriched genes in mESCs and mEpiSCs. FIG. 33 shows a UCSC browser image depicting the Kdm5b promoter. FIG. 34 illustrates expression levels (mean+/−SD) of pluripotency-enriched transcript Kdm5b and nonpluripotency-enriched genes in the mESC state, but not in the mEpiSC state. FIG. 35 shows aggregate plots of enhancers associated with the pluripotency-enriched genes in the mESC state, but not in the mEpiSC state. FIG. 36 shows boxplots depicting differences in the levels of enhancer histone marks between the two cell types as measured by RPKM (reads per kilobase per million mapped reads) at naïve-dominant enhancers and seed enhancers. FIG. 37 shows a percentage of pluripotency-enriched genes associated with naïve-dominant enhancers and seed enhancers in mESC Hi-C data sets. FIG. 38 shows a percentage of pluripotency-enriched genes for which both the gene target and an associated enhancer are bound by subunits of the mediator-cohesion complex (Med12, Nipb1, and Smc1).

FIGS. 39-48 illustrate how seed enhancers are utilized in downstream tissues where they expand into enhancer clusters. FIG. 39 shows a pie chart depicting the fraction of seed enhancers that show a chromatin state indicative of enhancer activity (H3K27ac+) in downstream tissues, FIG. 40 shows a heatmap displaying the downstream tissues in which each seed enhancer is active. FIG. 41 shows the percentage of seed enhancers active in at least one downstream tissue compared to naïve-dominant enhancers, MEF enhancers, and enhancers shared between mESCs and mEpiSCs. FIG. 42 shows the percent of seed enhancers active in each downstream tissue compared to naïve-dominant enhancers, MEF enhancers, and enhancers common between mESCs and mEpiSCs. FIG. 43 illustrates a genome browser image depicting the Cct3 gene and the enhancers predicted to regulate its transcription in mESCs and mEpiSCs. FIG. 44 illustrates that the expression (mean+/−SD) of Cct3 is high in pluripotent cells and embryonic brain, but low in bone marrow. FIG. 45 illustrates the percent of enhancers located in a cluster of enhancers (defined as four or more active enhancer elements within a 100 kb window) in a downstream tissue. FIG. 46 illustrates the percentage of seed enhancers, naïve-dominant enhancers, and all enhancers within a region that becomes an enhancer cluster in one of the four neural tissues in the panel (embryonic brain, cortex, olfactory bulb, and cerebellum). FIG. 47 is similar to FIG. 46, except for regions that are super enhancers in neural tissue. FIG. 48 illustrates motifs enriched among seed enhancers that are active in downstream tissues.

### DETAILED DESCRIPTION OF THE INVENTION

This disclosure relates to systems and methods that produce information that can indicate whether a gene is a target of an enhancer. As used herein, a “gene” can refer to a region of deoxyribose nucleic acid (DNA) that controls a discrete hereditary characteristic, usually corresponding to a single protein or ribonucleic acid (RNA). In some instances, the gene can include an entire functional unit, encompassing coding DNA sequences, non-coding regulatory DNA sequences, and introns. A gene can be “expressed” when an organism exhibits an observable phenotype that can be produced by the gene, often by directing the synthesis of a protein. Accordingly, a gene can be specifically expressed in a cell type when the organism associated with the cell type exhibits the associated observable phenotype. As used herein, an “enhancer” can be a DNA site to which one or more promoters (e.g., gene regulatory proteins) bind, influencing the rate of transcription of a gene. The enhancer can be located many thousands of base pairs (e.g., kilobase) either upstream or downstream from the gene that the enhancer influences. Accordingly, a gene can be a target of an enhancer when the enhancer influences the rate of transcription of the gene.

The systems and methods described herein can employ a bioinformanics algorithm to provide and/or utilize information that indicate whether a gene is a target of an enhancer. In some instances, the information can include a genomic proximity value indicating the proximity between the enhancer and the gene. In other instances, the information can include a value related to an expression level of the gene and a value related to a methylation level of the enhancer. In further instances, the information can include a relationship of the value related to an expression level of the gene, the value related to a methylation level of the enhancer, and values related to gene expression levels and enhancer methylation levels of a stored set of known enhancer-gene pairs. The information can be used to predict whether the gene is a target of the enhancer. As an example, the systems and methods can provide information that can be used to predict specific tissue interactions of genes and enhancers with high accuracy, specificity, and breath, requiring only enhancer data (e.g., H3K4me1 chromatin-immunoprecipitation-sequence, or ChIP-seq, data) and gene expression data.

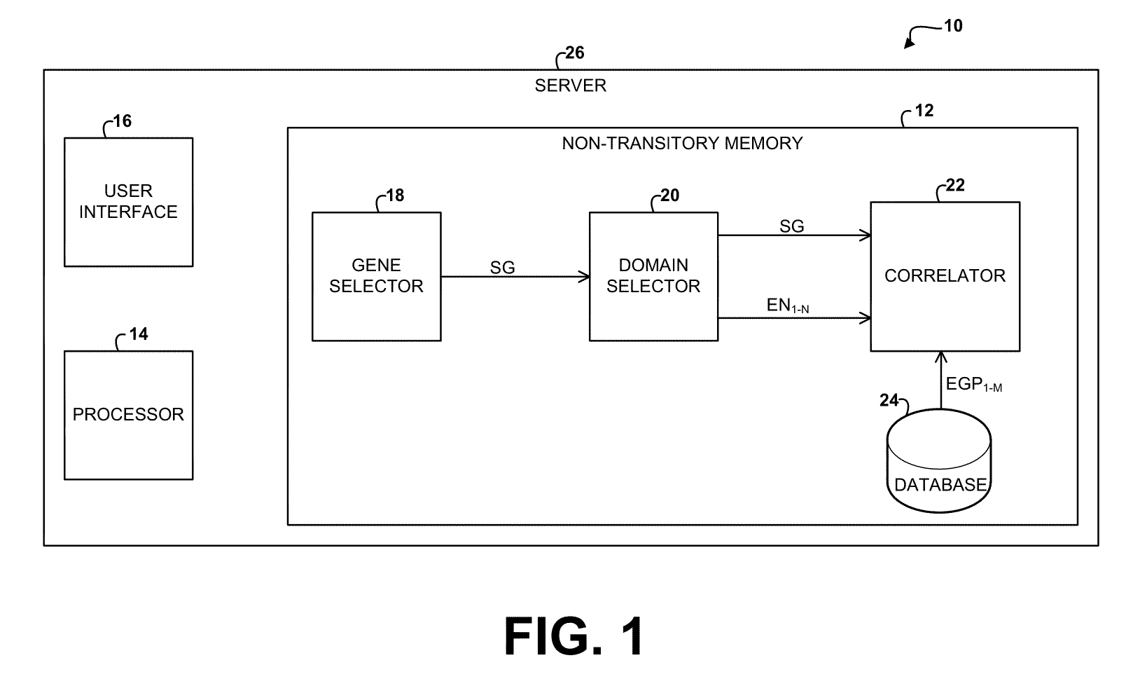

FIG. 1 illustrates an example of a system 10 that can provide information that can be utilized in the determination of whether a gene is a target of an enhancer in accordance with an aspect of the present disclosure. In some instances, system 10 can provide information for different cell types across the genome and is not restricted to specific cell types (e.g., corresponding to the set of known enhancer-gene pairs). In other instances, system 10 is not restricted to the human genome and can produce information for other species of animals. As illustrated, system 10 can be stored by a non-transitory memory 12 and executed by a processor 14 of a server 26. The server 26 can be replaced with any machine that includes a non-transitory memory the can store the machine-readable instructions corresponding to the system 10 and a processor that can access the non-transitory memory and execute the machine-executable instructions.

The system 10 can include a gene selector 18 that can receive an input of a gene (SG) from a given cell type. For example, the gene (SG) can be specifically expressed in the given cell type. As used herein, a specifically expressed gene can refer to a gene that is expressed preferentially in the given cell type compared to different cell types. In other words, while the specifically expressed gene may be expressed in another cell type, the specifically expressed gene can exhibit a much higher expression level in the given cell type compared to the other cell types. Although system 10 is described with receiving a single gene input, it will be understood that the input can include a plurality of genes and each component of system 10 can perform the same actions for each of the plurality of genes either sequentially or concurrently.

In some instances, gene (SG) can be input by a user via a user interface 16. In other instances, the gene (SG) can be selected from a plurality of genes provided in a list that is displayed to the user and selected via the user interface. After receiving the input of the gene (SG), the gene selector 18 can retrieve gene expression data (e.g., determined based on a transcription sequencing technology, such as RNAseq) corresponding to the gene (SG). In some instances, the gene expression data can be stored within the non-transitory memory 12. In other instances, the gene expression data can be entered by the user via the user interface 16. In still other instances, the gene expression data can be stored in a database located remotely from system 10. In still other instances, the gene (SG) can be determined through an automated process (e.g., via data mining the human genome for various expressed genes in a given cell type).

The system 10 can also include a domain selector 20 that can establish a domain across the gene (SG). For example, the domain can be selected to indicate a genomic proximity value between an enhancer within the domain and the gene. An enhancer within close genomic proximity to the gene can be thought of as a potential regulator of the gene.

In some instances, the domain can be established around a “start position” of the gene. For example, the “start position” can be a transcription start site (TSS) where the first step of gene expression occurs, in which the particular segment of DNA is copied into RNA by the enzyme RNA polymerase. The domain selector 20 can establish a preliminary domain that spans a certain number of kilobase (kb), where 1 kb=1000 base pairs, both upstream and downstream from the start position. It will be appreciated that the number of base pairs can be the same on either side of the established position, but need not be the same. Accordingly, in one example, the preliminary domain can span from 100 kb upstream from the start position to 100 kb downstream from the start position. While in another example, the preliminary domain can span from 200 kb downstream to 100 kb downstream from the start position.

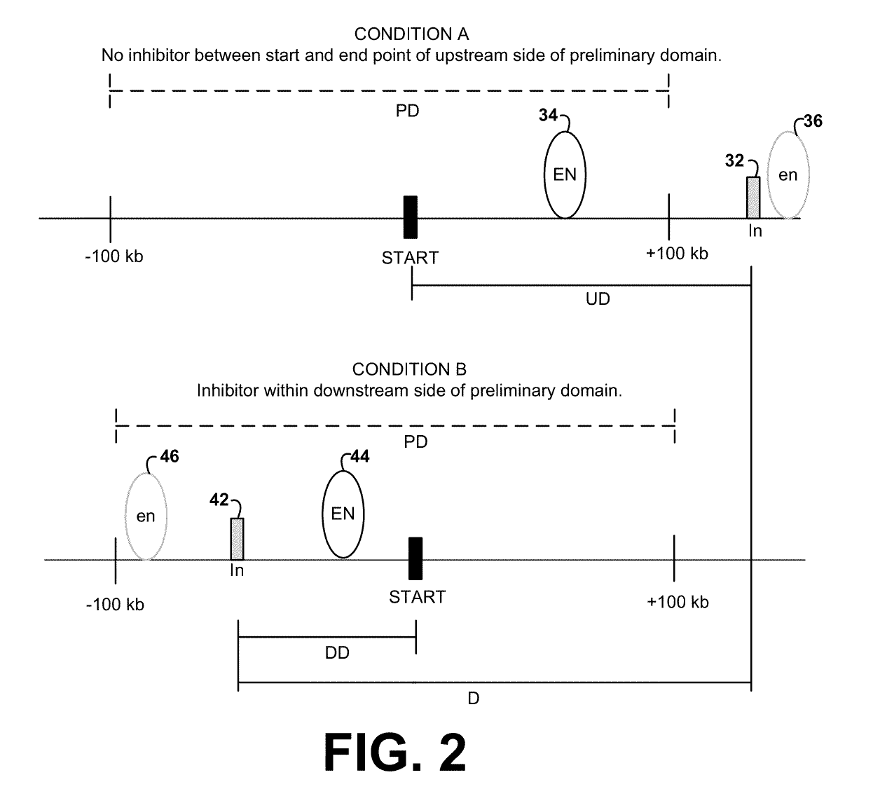

The domain selector 20 can use the preliminary domain to establish the domain according to two conditions related to a position of an inhibitor within the preliminary domain. In some cases, the inhibitor can be a CTCF binding site that can block the interaction between enhancers and promoters when CTCF binds to the binding site. The first condition relates to a case where no inhibitor is located within the preliminary domain, as shown in Condition A of FIG. 2. The second condition relates to a case where an inhibitor is located within the preliminary domain, as shown in Condition B of FIG. 2. For simplicity of explanation, the preliminary domain (PD) shown in FIG. 2 spans from 100 kb upstream (−100 kb) from the start position (START) to 100 kb downstream (+100 kb) from the start position (START). Condition A of FIG. 2 is illustrated on the downstream side of the preliminary domain (e.g., from START to +100 kb). Condition B of FIG. 2 is illustrated on the upstream side of the preliminary domain (e.g., from START to −100 kb). However, Conditions A and B are not limited to the illustrated respective upstream and downstream side. Both of conditions A and B can occur on one side, both sides, or neither side.

In Condition A, as illustrated in FIG. 2, no inhibitor is located within the upstream side of the preliminary domain (PD). In this case, the upstream side of the domain (UD) can extend beyond the end point of the preliminary domain (e.g., beyond +100 kb, as illustrated) until an inhibitor 32 is reached. Any enhancers located between the start position (START) and the inhibitor 32 can be considered a potential enhancer that can target the gene (e.g., enhancer 34). Any enhancer located beyond the inhibitor 32 can be excluded as a potential enhancer that can target the gene (e.g., enhancer 36).

In Condition B, an inhibitor 44 is located inside the downstream side of the preliminary domain (PD). In this case, the downstream side of the domain (DD) can end before the end point of the preliminary domain (e.g., before −100 kb, as illustrated). As illustrated, the downstream side of the domain (DD) can end at the location of the inhibitor 42 so that any enhancer located between the inhibitor and the start (START) can be considered a potential enhancer that can target the gene (e.g., enhancer 44) and any enhancer located beyond the inhibitor can be excluded as a potential enhancer that can target the gene (e.g., enhancer 46).

The domain (D) can be determined by combining the upstream domain (UD) and the downstream domain (DD). The domain (D) need not be the same length on both sides. As an example, the domain (D) can extend downstream to inhibitor 32, as shown in Condition A of FIG. 2, and on upstream to inhibitor 42, as shown in Condition B of FIG. 2. In this example, enhancer 34 and enhancer 44 can be considered as potential enhancers that can target the gene, while enhancers 36 and 46 can be excluded as potential enhancers for the particular gene. Although enhancers 36 and 46 can be excluded from being potential enhancers for the particular gene, these enhancers may still be identified as potential enhancers for different genes.

Referring again to FIG. 1, the domain selector 20 can output the selected gene (SG) and a set of potential enhancers (EN_{1-N}, where N is an integer greater than or equal to 1) identified within the domain. The set of potential enhancers (EN_{1-N}) can include any enhancer within the domain (or, in other words, within genomic proximity to the gene). For example, the set of enhancers can include enhancer 34 and enhancer 44, as shown in FIG. 2. The system 10 can also include a correlater 22 that can receive the selected gene (SG) and the set of potential enhancers (EN_{1-N}). Although the domain selector 20 is illustrated as acting before the correlater 22, the correlater can operate before the domain selector and/or in combination with the domain selector, or after the domain selector.

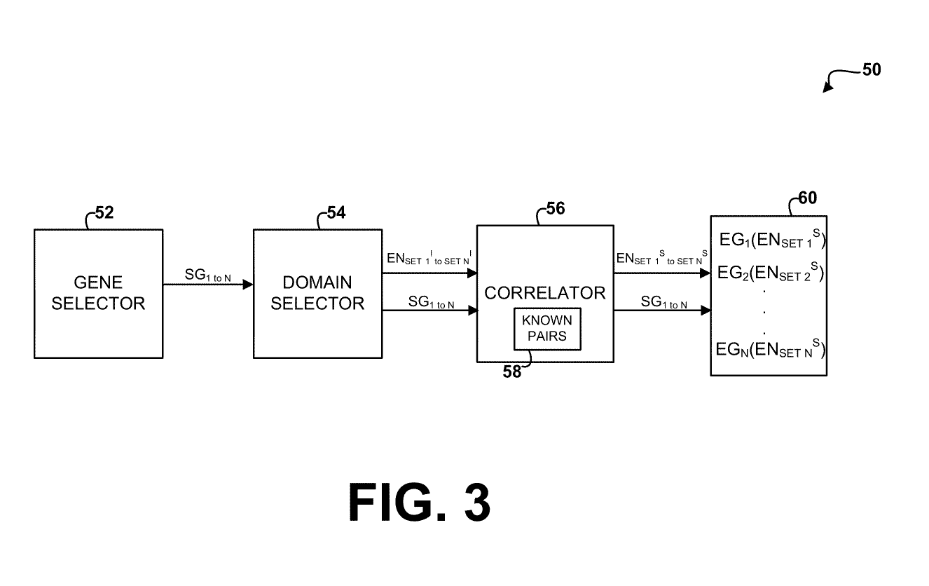

The correlater 22 can create one or more preliminary pairs of each of the set of enhancers and the selected gene. An example, as shown in FIG. 3, when the set of enhancers includes EN₁-EN_{N}, the set of preliminary pairs can include {SG-EN₁, SG-EN₂, . . . , SG-EN_{N}}. In the illustrated example, one of the set of preliminary pairs (e.g., SG-EN₁, which is circled) can be selected at a time for further processing.

As shown in FIG. 1, the correlater 22 can access a database 24 or other storage medium (either within the server 26 or remote from the server) to retrieve data related to a known set of enhancer-gene pairs (EGP_{1-M}, where M is an integer greater than or equal to 1) Although a set of one known enhancer-gene pair can be used by system 10, the more known enhancer-gene pairs are used, the more accurate the information provided by system 10.

The correlater 22 can compare each of the set of preliminary pairs to the known set of enhancer-gene pairs (EGP_{1-M}). As shown in FIG. 3, the comparison can be done by comparator 52, which takes inputs of the proposed pair SG-EN₁ and the known set of enhancer-gene pairs (EGP_{1-M}) and can output information that can indicate a chance the gene (SG) is a target of the tested enhancer (ENO. The comparator 52 can compare values related to the gene (SG Value) to values related to the genes in the known set of enhancer-gene pairs (EGP_{1-M}). The comparator can also compare values related to the enhancer (EN Value) to values related to the enhancers in the known set of enhancer-gene pairs (EGP_{1-M}).

As an example, the values related to the gene (SG Value) can include an expression level value that can indicate whether the gene is specifically expressed in the cell type. The comparator 52 can compare the expression level value of the gene to corresponding expression level values of the genes in the set of known enhancer-gene pairs (EGP_{1-M}). In some examples, the comparator 52 can compare data related to the expression level of the gene to the corresponding expression level value of the genes in the set of known enhancer-gene pairs based on a predetermined first threshold. As an example, the expression level values can be based on or include Shannon entropy data (including Shannon entropy Q value data, where Q represents the amount of heat put into the system) and/or data related to a methylation level of the gene. In one example, the predetermined first threshold can be related to the Shannon Entropy Q value according to a high stringency condition, in which the predetermined first threshold can be value that maximizes the positive selection rate (e.g., a Shannon Entropy Q score less than or equal to about 6). In another example, the predefined first threshold can be related to the Shannon Entropy Q value according to a low stringency condition in which the predetermined first threshold can be a value that maximizes the total number of predictions while maintaining an estimated positive prediction rate greater than 60 percent (e.g., a Shannon Entropy Q score less than or equal to about 6.8).

As another example, the values related to the enhancer (EN Value) can include data related to a methylation level of the enhancer. The comparator 52 can compare data related to the methylation level of the enhancer to corresponding data related to the methylation level of the enhancers to corresponding methylation level values of the enhancers in the set of known enhancer-gene pairs (EGP_{1-M}) pairs based on a predetermined second threshold. As an example, the data related to the methylation level can be based on or include Shannon entropy data (including Shannon entropy Q value data, where Q represents the amount of heat put into the system). In one example, the predetermined second threshold can be related to the Shannon Entropy Q value according to a high stringency condition, in which the predetermined second threshold can be value that maximizes the positive selection rate (e.g., a Shannon Entropy Q score less than or equal to about 6). In another example, the predefined second threshold can be related to the Shannon Entropy Q value according to a low stringency condition in which the predetermined first threshold can be a value that maximizes the total number of predictions while maintaining an estimated positive prediction rate greater than 60 percent (e.g., a Shannon Entropy Q score less than or equal to about 6.1).

Based on the comparison, the comparator 52 can output information that can be utilized to determine the chance the gene (SG) is a target of the enhancer (e.g., EN₁). Referring again to FIG. 1, in some examples, the correlater 22 can output the information that can be utilized to determine the chance the gene (SG) is a target of the enhancer (e.g., EN₁) to a display. In other examples, the correlater 22 can predict whether the gene (SG) is a target of the enhancer (e.g., EN₁) based on the information that can be utilized to determine the chance the gene (SG) is a target of the enhancer (e.g., EN₁). In this example, the correlater 22 can output the prediction to the display. In some examples, the prediction can be output to the display in connection with the information that can be utilized to determine the chance the gene (SG) is a target of the enhancer (e.g., EN₁).

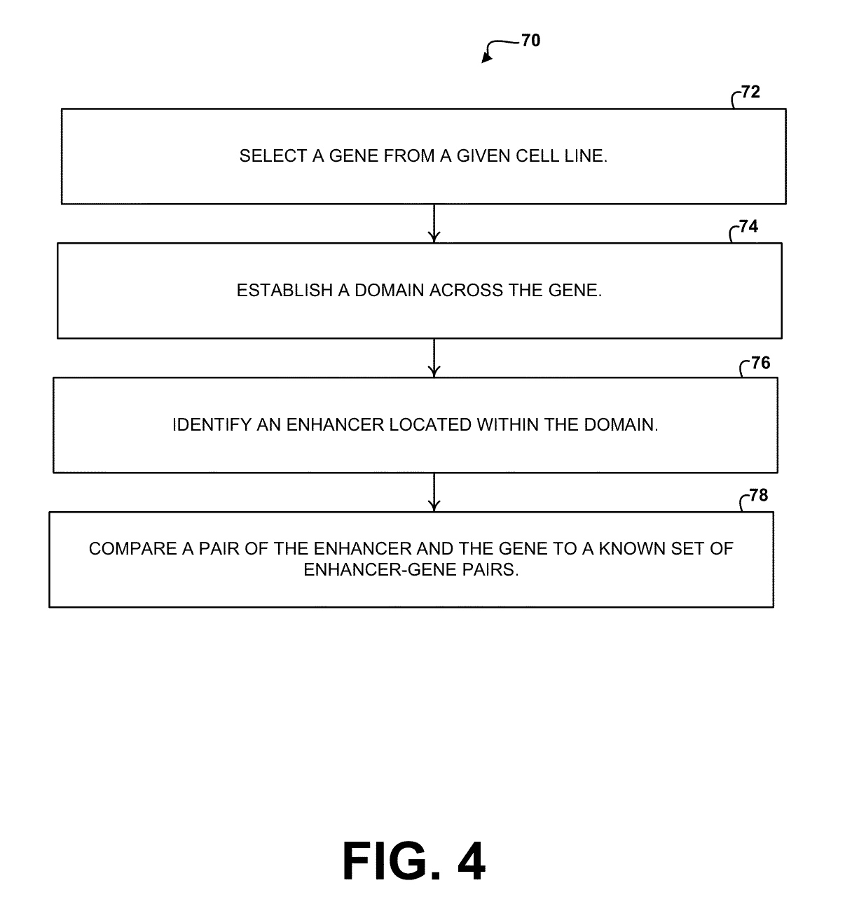

In view of the foregoing structural and functional features described above, example methods will be better appreciated with reference to FIG. 4. While, for purposes of simplicity of explanation, the method of FIG. 3 is shown and described as executing serially, it is to be understood and appreciated that the present invention is not limited by the illustrated order, as some actions could, in other examples, occur in different orders from that shown and described herein or could occur concurrently. It will be appreciated that some or all of each of these methods can be implemented as machine-readable instructions on a non-transitory computer readable medium.

FIG. 4 illustrates an example of a method 70 for determining information that indicates whether a gene is a target of an enhancer. The information can facilitate the prediction of gene targets of enhancers. The method 70 can provide accurate information in an efficient manner can be based on genomic proximity between the enhancer and the gene and a comparison to known enhancer-gene pairs. At element 72, a gene can be selected from a given cell type (e.g., by gene selector 18). At element 74, a domain can be established across the gene (e.g., by domain selector 20). At element 76, one or more enhancers located within the domain can be identified (e.g., by domain selector 20). The enhancers located within the domain can exhibit a genomic proximity to the gene. At element 78, a pair of one of the identified enhancers and the gene can be compared to a known set of enhancer-gene pairs (e.g., by correlater 22). The information related to the comparison can be utilized in connection with information related to the genomic proximity to determine whether the gene is a target of the enhancer.

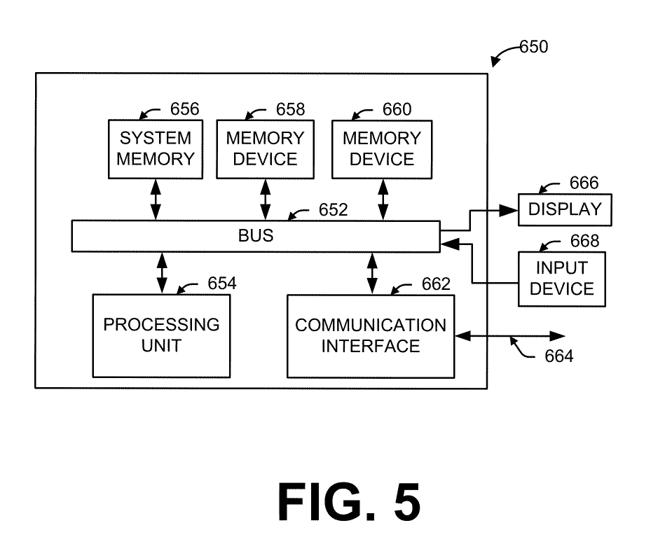

FIG. 5 is a schematic block diagram illustrating an example system 650 (that can, for example, include server 26) that includes a system of hardware components capable of implementing examples of the systems and methods disclosed in FIGS. 1-4 to provide information that can be used in the prediction of gene targets of enhancers based on genomic proximity and a comparison to a set of known enhancer-gene pairs. The system 650 can include various systems and subsystems. The system 650 can be a personal computer, a laptop computer, a workstation, a computer system, an appliance, an application-specific integrated circuit (ASIC), a server, a server blade center, a server farm, etc.

The system 650 can include a system bus 652, a processing unit 654, a system memory 656, memory devices 658 and 660, a communication interface 662 (e.g., a network interface), a communication link 664, a display 666 (e.g., a video screen), and an input device 668 (e.g., a keyboard and/or a mouse). The system bus 652 can be in communication with the processing unit 654 and the system memory 656. The additional memory devices 658 and 660, such as a hard disk drive, server, stand-alone database, or other non-volatile memory, can also be in communication with the system bus 652. The system bus 652 interconnects the processing unit 654, the memory devices 656-660, the communication interface 662, the display 666, and the input device 668. In some examples, the system bus 652 also interconnects an additional port (not shown), such as a universal serial bus (USB) port.

The processing unit 654 can be a computing device and can include an application-specific integrated circuit (ASIC). The processing unit 654 executes a set of instructions to implement the operations of examples disclosed herein. The processing unit can include a processing core.

Like the system memory 656, the additional memory devices 658 and 660 can store data, programs, instructions, database queries in text or compiled form, and other information necessary to operate a computer. The memory devices 656, 658 and 660 can be implemented as computer-readable media (integrated or removable) such as a memory card, disk drive, compact disk (CD), or server accessible over a network. In certain examples, the memory devices 656, 658 and 660 can comprise text, images, video, and/or audio, portions of which can be available in formats comprehensible to human beings.

In some instances, the system 650 can access an external data source or query source through the communication interface 662, which can communicate with the system bus 652 and the communication link 664.

In operation, the system 650 can be used to implement one or more parts of a predictive modeling system in accordance with the present invention. Computer executable logic for implementing the predictive model resides on one or more of the system memory 656, and the memory devices 658, 660 in accordance with certain examples. The processing unit 654 executes one or more computer executable instructions originating from the system memory 656 and the memory devices 658 and 660. The terms “computer readable medium” and “computer readable storage medium” as used herein refers to a non-transitory medium (e.g., not a transitory signal) that participates in providing instructions to the processing unit 654 for execution.

### EXAMPLES

The following examples are for the purpose of illustration only and are not intended to limit the scope of the appended claims.

### Example 1

This example shows cell type-specific enhancer-gene interactions can be accurately identified using the systems and methods described herein. In this example, the systems and methods are executed by the software program, PreSTIGE (Predicting Specific Tissue Interactions of Genes and Enhancers).

### Methods

### PreSTIGE Methodology

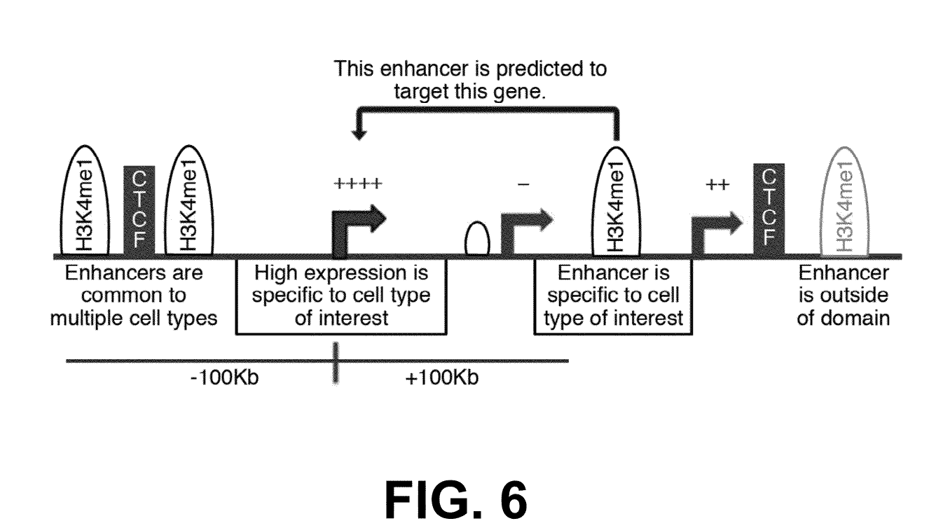

PreSTIGE integrates available H3K4me1 ChIP-seq and RNA-seq datasets from a panel of 12 diverse cell types, and then pairs the cell type-specific H3K4me1 signals with genes that are generally specifically expressed in each cell type. The prediction scheme utilizes a “combined” linear domain model that considers ChIP-seq identified CTCF binding sites, a subset of which demarcate insulator elements, in addition to distance between transcription start sites (TSSs) and enhancers (FIG. 6). This model considers only “common” CTCF binding sites detected in more than 20 cell types. Only CTCF sites >100-kb from a TSS are considered as enhancer blockers. The 100-kb distance is based on analyses indicating that H3K4me1-sites are most enriched within 100-kb of the TSSs of genes that are specifically expressed in a given cell type.

Domain models that spanned greater distances (≧500-kb) were also considered, but that the enrichment of H3K4me1 sites is greatly depleted at distances beyond 100-kb. Rarefaction curves (saturation plots) were generated to determine the fraction of all possible expressed genes, and all possible enhancers detected using these 12 cell types. These plots demonstrate little increase of transcripts and enhancers occurring upon addition of the 12th cell type. Therefore, the enhancers and transcripts among the 12-cell type panel are of sufficient diversity and complexity for comparative purposes, and that the addition of lines to the panel would only marginally improve the prediction model.

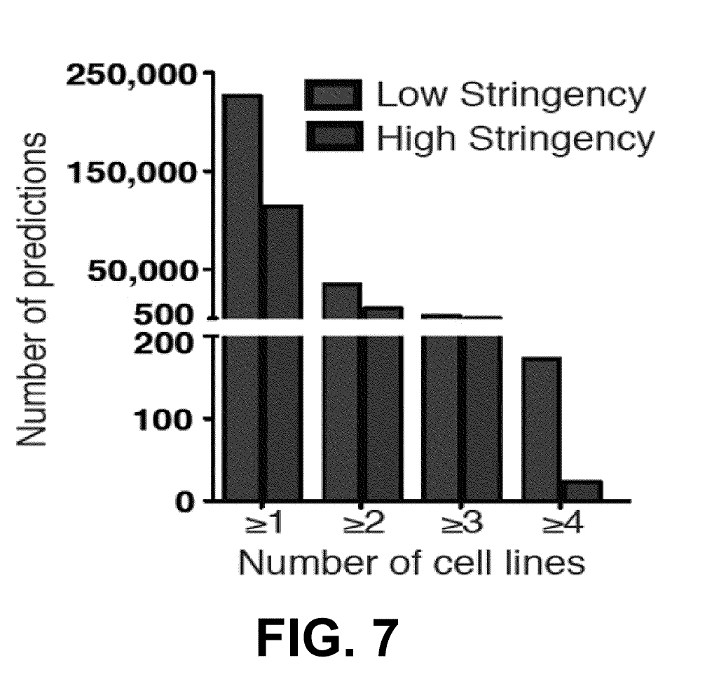

Each enhancer and each transcript in all 12 cell types were assigned specificity scores using a function of Shannon entropy. For each cell type, specific enhancers were then paired with specifically expressed genes contained within PreSTIGE-defined domains. Enhancer-gene assignments were made using two different specificity thresholds: low and high. These thresholds were methodically defined by balancing the positive predictive rate with the total number of interactions predicted. Quantitative specificity scoring resulted in over 226,000 low stringency and 113,000 high stringency predictions made across the 12 cell types, 15% of which were predicted to occur in more than 1 cell type (FIG. 7). Enhancers used in the predictions were found to be significantly enriched for H3K27ac, which has been shown to mark active gene enhancer elements. Thus, PreSTIGE “selects” for active enhancer elements without requiring the addition of H3K27ac ChIP-seq profiles to the prediction scheme.

### ChIP-Seq Data Processing

Publically available H3K4me1 ChIP-seq and matched input data files were obtained for the 12 cells lines of the comparator set and aligned to hg18 with BWA. Duplicate reads were removed with SAMtools. Matched inputs for each sample were trimmed to 10 million reads prior to alignment and used for peak-calling with MACS. Called peaks were used to generate a list of potential enhancer sites. All identified ChIP enriched peaks across the 12 cell types were then compiled and overlapping peaks were collapsed resulting in 309,713 regions. The maximum signal was then retrieved in each region across all 12 cell types and the results were tabled. To normalize for read depth and varying enrichment across ChIP samples, maximum signals were quantile normalized. Shannon Entropy scoring was performed on normalized maximum signals to quantify cell-line specificity for each region.

### RNA-Seq Data Processing

Publically available RNA-seq data were obtained for all 12 cell types of the comparator set. Reads were aligned to hg18 with TopHat allowing for a maximum of 10 multiple hits. Gene expression score FPKM (fragments per kilobase per million reads) was determined for all refseq genes using Cufflinks. An FPKM threshold of 0.3 was chosen to balance the false discovery and false negative rates. Genes with FPKMs below 0.3 were rounded to zero and then the results were tabled. The data obtained for Neural Precursor Cells (NPCs) was sequenced on the ABI solid platform, and was aligned using TopHat modified for colorspace reads. Given the different platforms used in sequencing the 12 samples, FPKMs were quantile normalized. Shannon Entropy scoring was then performed on the normalized FPKMs to score cell type specificity of gene expression.

### PreSTIGE Prediction Methodology

All potential interacting enhancer-gene pairs were identified within the specified domains. Domains include all enhancers with 100 kb of the TSS and all enhancers within the CTCF domain containing the TSS. For an interaction to be predicted for a given cell type, the normalized H3K4me1-enhancer signal had to be high above background (>10). For a potential interacting enhancer-gene pair to be predicted in a given cell type, both the enhancer and the gene have to be specific to the cell type. Two levels of stringency were used to define specificity. For high stringency predictions the Shannon Entropy Q score had to be below 6 (the lower the Q score the greater the specificity to a given line) for the enhancer and below 6 for the gene paired to that enhancer. For low stringency predictions the Q score had to be below 6.1 for the enhancer and below 6.8 for the gene paired to that enhancer. These thresholds were defined based by balancing the positive prediction rate and the total number of predictions made. The positive prediction rate was calculated as described below (FDR Calculation with CRC VEL data), for several different specificity thresholds. For each specificity threshold, the number of predictions made in each cell was also determined. The two thresholds were selected, high stringency which maximizes the positive prediction rate and low stringency which maximizes the number of predictions made while maintaining an estimated positive prediction rate >60%.

### H3K27ac Analysis

H3K27ac ChIP-seq data were obtained from publically available datasets (see Table S3). ChIP-seq data was processed as described above. The H3K27ac signal was obtained in 10 kb windows surrounding the midpoint of the H3K4me1 peaks of the comparator set using 50 bins. These results were then z-scored. The average signal in each bin was determined for the HepG2 specific peaks, all HepG2 peaks (those region with maximum signal over 10 in HepG2) and all enhancer regions of the comparator set. Paired T-test was performed comparing the H3K27ac signal in the 10 center bins of HepG2 specific enhancers vs. all HepG2 enhancers (p-value<3.4 E-5). This was repeated for GM12878 and H1ES cells and all three cell types displayed similar results.

### Canonical Enhancer Validation

16 previously discovered enhancer-gene interactions were identified from the primary literature that involve a gene that is specifically expressed in one of the 12 cell types of the panel. These interactions were evaluated with the specificity stringencies described above to determine whether the PreSTIGE methodology predicts the known interaction. For an enhancer to be validated, it had to be within 2 kb of the enhancer region found within the comparator set. (i.e. if the identified enhancer is described as 40 kb upstream of the TSS and the enhancer present in the comparator set overlaps with the region 38-42 kb upstream then this enhancer is considered for validation). All previously identified interactions were evaluated with the specificity stringencies described above to determine whether the PreSTIGE methodology predicts the known interaction.

### ChIA-PET Validation

Enhancer-promoter interactions previously identified through ChIA-PET were utilized for validating PreSTIGE predictions within the 12-cell type comparator set. All interactions identified in each biological replicate were considered for validation. These interactions involve two loci (anchors), one of which is within 5 kb of a TSS. The remaining anchor region was intersected with the enhancer regions found in the comparator set. All ChIA-PET interactions considered for validation contained one anchor within 5 kb of a TSS and the other anchor overlapped with any of the 309,713 enhancer regions found in the comparator set. Next the number of PreSTIGE predictions that were found within the set of ChIA-PET identified enhancer-gene interactions were determined and considered these predictions validated by ChIA-PET. To determine the enrichment of cell type-specific predictions in both the MCF-7 and K562 datasets the odds ratio was calculated. The total number of predictions made for the cell type were compared to the number of predictions validated by ChIA-PET. This was compared to the total number of candidate interactions that are not predicted to occur in the cell type (i.e. those enhancer-gene pairs that are within the specified domains but are not specific to the cell type) that validated by ChIA-PET. The odds ratios for predictions made in each of the 12 cell types were calculated using the following equation: OR=(True Positives/False Positives)/(False Negatives/True Negatives); True Positives-Predications validated by ChIA-PET; False Positives-Predictions not validated; False Negatives-Interactions not predicted by PreSTIGE but are found by ChIA-PET; True Negatives-Interactions not predicted that are not found by ChIA-PET.

eQTL Validation

eQTL data was obtained from the GTEx (Genotype-Tissue Expression) eQTL Browser from both liver and lymphoblastoid studies. To be considered for validation the eQTL had to fulfill the following criteria: the SNP had to fall within a cell type-specific enhancer, the gene had to be 5-125 kb from the SNP and the P value for the eQTL association had to be less than 0.001. It was next determined which of these eQTLs were predicted by PreSTIGE. The percentage of PreSTIGE predicted eQTLs were compared to the percent expected to validate by random chance by randomly pairing enhancers with genes contained within the same domain, and randomly pairing cell type-specific enhancers with genes within the PreSTIGE defined domains. Chi-squared was utilized to compare the enrichment of PreSTIGE predictions to both randomizations.

### SC Validation

5C interactions were obtained for 3 cell types GM12878, K562 and HeLa. Restriction fragment pairs that were found to interact (q<0.01) in both biological replicates were considered looping interactions and restriction fragment pairs that were not found to interact (q>0.01) in either biological replicate were considered as non-looping pairs. The fraction of looping and non-looping pairs predicted by each method (PreSTIGE low and high stringency, nearest gene and nearest expressed gene) was determined. The enrichment ratio was calculated by dividing the percent of looping pairs predicted by the percent of non-looping pairs predicted.

### Colon Cancer VEL Data Validation

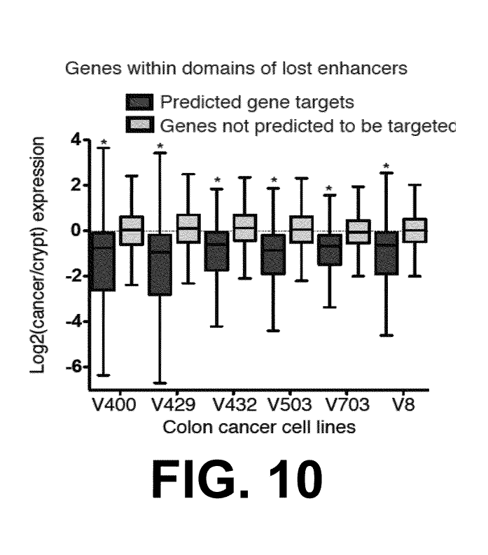

H3K4me1 ChIP-seq and matching input for the colon crypt was processed as described above. The crypt peaks were added to the 309,713 peaks of the comparator set and peaks within 1 by were consolidated. The maximum signal in all regions was obtained for all 13 cell types and the results were tabled and quantile normalized. These results were then Shannon Entropy scored. Previous exon array data for healthy colon crypt and colon cancer cell types was also obtained. The median PLIER normalized expression score across 3 biological replicates of the colon crypt was used in subsequent analysis. To accurately compare colon crypt array data to the RNA-seq data of the comparator set, array expression was quantile normalized with the RNA-seq data table and then Shannon Entropy scoring was used to quantify specificity of gene expression. Predictions were made as described above for interactions that occur in the colon crypt and CRC samples. For validation of the crypt predictions, colon cancer cell types were analyzed in pairs. H3K4me1 sites that show differential enrichment of H3K4me1 between crypt and cancer (i.e. lost VELs) and those that are defined as unchanged, present in the crypt and cancer lines, were considered for validation. As the distributions of expression were different between the 6 colon cancer cell types and the median colon crypt expression, datasets were quantile normalized to control for any bias toward increase or decrease in gene expression between the cancer cell types and the crypt due to differences in distributions. Mann-Whitney-Wilcoxon test was used to compare expression (relative to the crypt) of predicted gene targets to genes within the domain of a lost enhancer that are not predicted to be targeted (FIG. 10) as well as to compare expression of gene targets of lost enhancers to gene targets of control enhancers.

FDR Calculation with CRC VEL Data

Fold changes of transcripts in colon cancer versus normal colon crypts were calculated (CRC/median of 5 normal crypt samples). Enhancer-gene predictions were made in the normal colon crypts and used to determine the gene targets of enhancers lost, i.e., lost VELs, in the colon cancer cell types. If the enhancer was lost and the expression of the predicted gene targeted decreased more than 1.3 fold, then the enhancer-gene pair was considered successfully validated. Genes associated with a lost VEL that failed to show a decrease in gene expression in CRC by more than 1.3 fold were considered false positives.

### Annotation of Non-Coding GWAS Variants

The entire NHGRI catalog of GWAS variants was downloaded, and all SNPs in LD with GWAS lead SNPs using LD blocks were identified with publically available HapMap data on the CEPH ancestry population. SNPs in strong LD (LOD>2 and D′>0.99) with the lead SNP were utilized. All lead and LD SNPs were intersected with human coding exons obtained from UCSC table browser. If the lead SNP or any of its LD SNPs intersected with the coding sequences that lead SNP (and its LD SNPs) were removed from the analysis. All subsequent analyses utilized the identified non-coding GWAS SNPs.

Variant Set Enrichment Analysis was used to test for enrichment of immune-related disorders in B-cell enhancers. SNPs associated with one of the 6 disorders (rheumatoid arthritis, Crohn's disease, multiple sclerosis, systemic lupus, ulcerative colitis and celiac disease) were intersected with the PreSTIGE predicted enhancers for all 12 cell types of the comparator set as well as the colon crypt. To determine if enrichment of SNPs in a given cell type is statistically significant, null distributions were generated by randomly sampling variants from Illumina HumanOmniExpress SNP list. Random SNP sets were matched to disease-associated SNP by size so that SNPs in the random set contained the same number of LD SNPs as the disease-associated set. Enrichment in PreSTIGE predicted enhancers of Disease-associated-SNP and 1000 random size-matched sets were compared in order to obtain the significance of the enrichment.

To determine the effect of the risk variant on the expression of the predicted gene target, RNA-seq gene expression data 61 CEU individuals as well as the corresponding genotypes from were obtained from HapMap. Using RA and CD disease associated SNPs, individuals who were homozygous for the non-risk allele, heterozygous for the risk allele and homozygous for the risk allele were identified. Any SNP for which fewer than five individuals had the minor allele was excluded from the analysis. The gene expression of individuals who carried the risk allele (homozygous or heterozygous) were compared to those who were homozygous for the non-risk variant using Mann-Whitney-Wilcoxon test (p-value<0.05).

To evaluate the specificity of GWAS enhancer variants and predicted gene targets, Shannon entropy scoring was utilized and the entropy (Q) scores of H3K4me1 sites identified via MACS in the GM12878 cell type were compared to those of the H3K4me1 sites that intersect with disease-associated SNPs. Two of the six of the diseases, multiple sclerosis and celiac disease, were significantly more cell type specific (Wilcox-test p-value<0.05) than all GM12878 enhancers. Next, the specificity of predicted gene targets was evaluated. As a control, the entropy (Q) scores were determined for all genes associated with a PreSTIGE prediction in GM12878 and compared to the entropy (Q) scores for genes that are predicted to be targeted by a disease associated enhancer variant (Wilcox-test p-value<0.05).

### Multiple Enhancer Variants Analysis

GWAS SNPs associated with 6 traits, RA, CD, MS, ulcerative colitis, celiac disease and systemic lupus in the European population were utilized for this analysis. GWAS SNPs that fall within GM12878 enhancers associated with a gene target were identified by PreSTIGE (at high stringency) and determined what percentage of GWAS enhancer variants had SNPs in LD that fall within an additional enhancer predicted to target the same gene (at high stringency). LD SNPs were retrieved as described above, annotation of non-coding GWAS variants). As a control CEU SNPs that fall within GM12878 enhancers associated with PreSTIGE predictions (at high stringency) were identified and all SNPs that have previously been associated with disease were excluded. The percent of these control SNPs that are associated with multiple enhancer variants in LD were determined and a matched number of controls were randomly selected 100 times. The average of 100 iterations was utilized in Fisher's exact test comparing disease to nondisease rate of multiple enhancer variants.

### Results

### Calculation of FDR for PreSTIGE

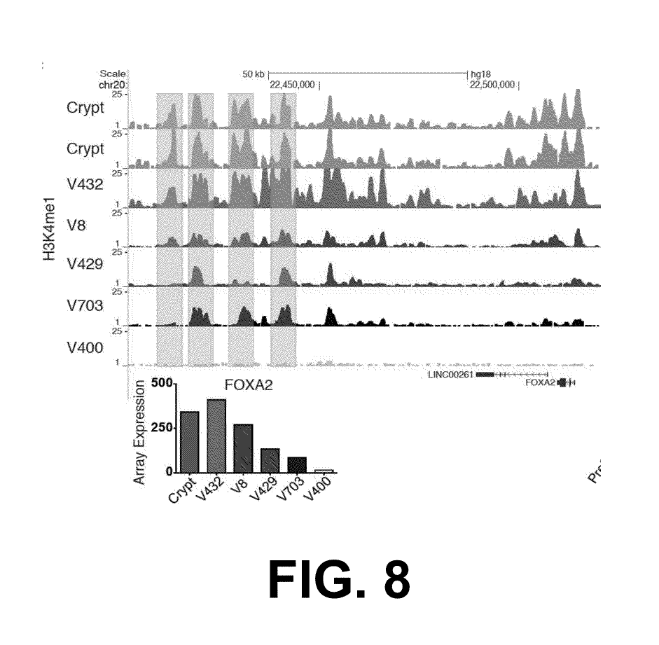

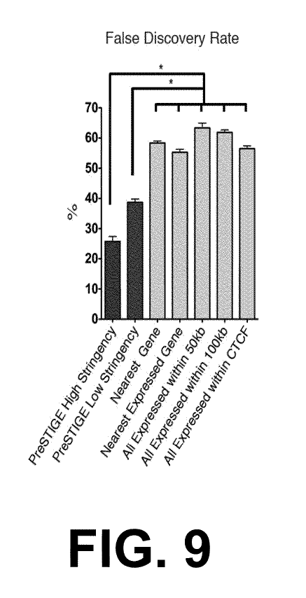

It was previously reported that colorectal cancer (CRC) cells harbor thousands of Variant Enhancer Loci (VELs), defined by locus specific gains and losses of H3K4me1 compared to normal colonic crypt specimens, from which CRC is derived. Similar to a knock-down or knock-out experiment, the loss of the H3K4me1 enhancer (lost VEL) would be expected to decrease the expression of the associated gene target. Conversely, a gain of the H3K4me1 signal (gained VEL) would be expected to increase expression of the associated gene target. Thus, it was reasoned that VELs could be utilized to estimate the accuracy of PreSTIGE predictions and assign a FDR. H3K4me1 ChIP-seq and transcriptome profiling was previously performed on 3 normal colon crypt samples and 9 CRC cell types. Using these data enhancer-gene predictions were generated in normal colon crypt and identified instances in which crypt enhancers were lost (i.e., lost VELs) in each CRC cell type. The expression of the crypt-predicted target transcript was then examined in both the normal colon crypt and the CRC sample in which the lost VEL was detected. It was thought that if an enhancer is present in the normal colon crypt, but absent in a CRC cell type, then an accurately predicted gene target of the enhancer would show reduced expression in the CRC line relative to the crypt. An example is shown in FIG. 8, where four enhancers in normal colon crypt are predicted to target FOXA2. In the CRC cell type V432, all four enhancers are present and accordingly, FOXA2 transcript levels are comparable between V432 and the normal colon crypt samples. In contrast, all four enhancers are “lost” in CRC sample V400, FOXA2 is reduced in V400 compared to normal crypts. The CRC lines V429 and V703 have 1-2 lost enhancers and expression of FOXA2 in these lines is also reduced (FIG. 8, bottom). To determine a FDR this analysis can be expanded globally by considering all predicted gene targets of lost enhancers that do not decrease in expression in CRC (>1.3-fold) relative to the normal colon crypt as false discovery events. This analysis yielded an FDR of 25% for high stringency PreSTIGE predictions, and 38% for low stringency PreSTIGE predictions. These estimates are likely conservative. First, the analysis fails to take into account genetic mutations or epigenetic events besides VELs that can affect transcript levels. Second, the analysis is based on the assumption that the loss of a single H3K4me1-enhancer always influences the expression of the target gene. Given that most genes are regulated by multiple enhancers, the loss of more than one enhancer may be required for a measurable decrease in transcript levels. In fact, the FDR drops to 18-32% when considering genes associated with two or more lost enhancers, and 13-23% for genes associated with three or more lost enhancers. Remarkably, only 42% of genes located nearest to lost VELs decreased in expression (approximating a FDR for the nearest-gene method at 58%). Furthermore it was determined the FDR for four additional methods commonly used to assign enhancers to genes (FIG. 9). The FDR ranges from 55-63% when the nearest expressed gene is predicted, when all expressed genes within 50 kb or 100 kb are predicted, and when all expressed genes in the CTCF domain are predicted. Thus, predictions made with PreSTIGE represent a 1.5 to 4.5-fold improvement over these common methods. The addition of H3K27ac to the prediction model did not significantly improve the FDR.

The VEL approach was next used to further evaluate the domain model. Enhancer-gene interactions separated by more than 100-kb validated at the same rate as those within 100-kb, indicating that extending domains from 100-kb to the nearest CTCF site increases the overall number of predictions without increasing the FDR. The VEL approach was also used to test enhancers predicted to target more than one gene (24% of crypt enhancers). 50-64% of enhancers predicted to target multiple genes validated, compared to 79-82% of interactions in which the enhancer was predicted to target only one gene. While these data suggest the most accurate predictions involve enhancers associated with one target gene, a significant proportion of enhancers within the genome target multiple genes.

Predicted targets of lost VELs were expressed at lower levels relative to normal colon crypts than genes that share the domain with the lost enhancer but are not predicted targets (FIG. 10). Targets of crypt enhancers lost in CRC were significantly repressed in CRC compared to a set of control genes for which predicted enhancers have comparable H3K4me1 levels between CRC and the crypt. These findings validate that the effect lost VELs on gene expression is specific to the gene predicted by PreSTIGE and not a general effect on all nearby genes.

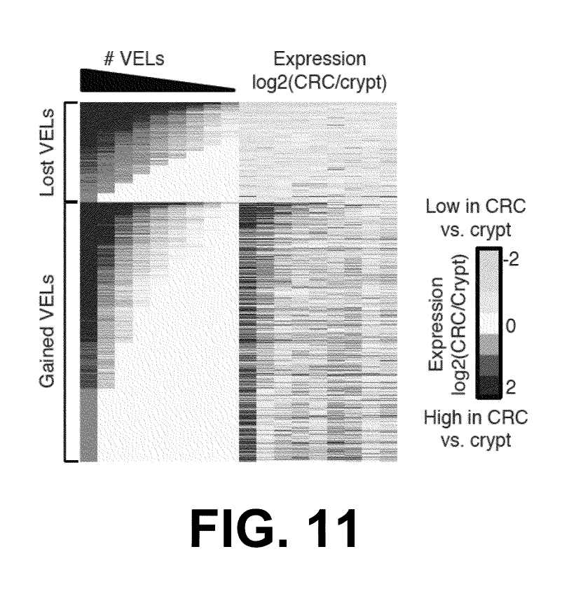

Lastly, as an additional qualitative assessment of the FDR, the targets of lost VELs in the colon crypt were predicted and their expression plotted in all nine CRC lines. Additionally the gene targets of the gained VELs were predicted in each of the CRC lines and the expression in the CRC lines relative to the crypts were plotted (FIG. 11). The heatmap reveals remarkable correlation between predicted genes and the presence or absence of the VEL, further suggesting that the FDR of the PreSTIGE method is low.

### Validation of PreSTIGE Predictions Amongst the 12-Cell Type Panel

The enhancer-gene assignments in the 12-cell type panel were further validated by first investigating 16 enhancer-gene interactions previously identified by 3C or other experimental methods. Remarkably, PreSTIGE correctly predicted 14 of these 16 interactions including interactions with APOB, APOE, FoxD3 and SOX2, DES, HBB, and HBA. 15/16 interactions fell within the PreSTIGE defined domains, while only 5/16 interactions would have been predicted if all CTCF sites were considered to have enhancer-blocking activity.

To validate on a more global scale, the predicted interactions were compared to a reference set of enhancer-promoter interactions identified through RNAPII ChIA-PET studies in MCF-7 and K562 cells. PreSTIGE predictions in K562 and MCF-7 cells were not only significantly enriched among ChIA-PET-identified interactions, but predictions made in other cell types were depleted from the ChIA-PET identified K562 and MCF-7 interactions. To further verify the enhancer-gene assignments among the reference set, expression Quantitative Trait Loci (eQTL) data from the GTEx Browser from liver and B lymphoblast cells were utilized. Remarkably, PreSTIGE predicted 35-54% of the eQTL determined enhancer-gene interactions, which is significantly higher than expected by random chance.

Finally, to validate predictions made in the comparator set, the predictions were compared to publically available 5C data. 5C experiments evaluate a defined set of restriction fragments for a given region of the genome. The enrichment of PreSTIGE predictions in looping interactions (true positive enrichment) were compared to enrichment of predictions in non-looping interactions (false positive enrichment) as identified by 5C. Both the nearest gene and nearest expressed gene prediction methods were compared to PreSTIGE by determining the enrichment ratio (true positive enrichment/false positive enrichment). Importantly, while all three methods showed some enrichment in looping interactions, when the false positive rate is considered, PreSTIGE clearly out performs the other two methods. Thus PreSTIGE predictions share high concordance with experimental methods aimed at determining enhancer-gene interactions.

### Outcome of PreStige Model

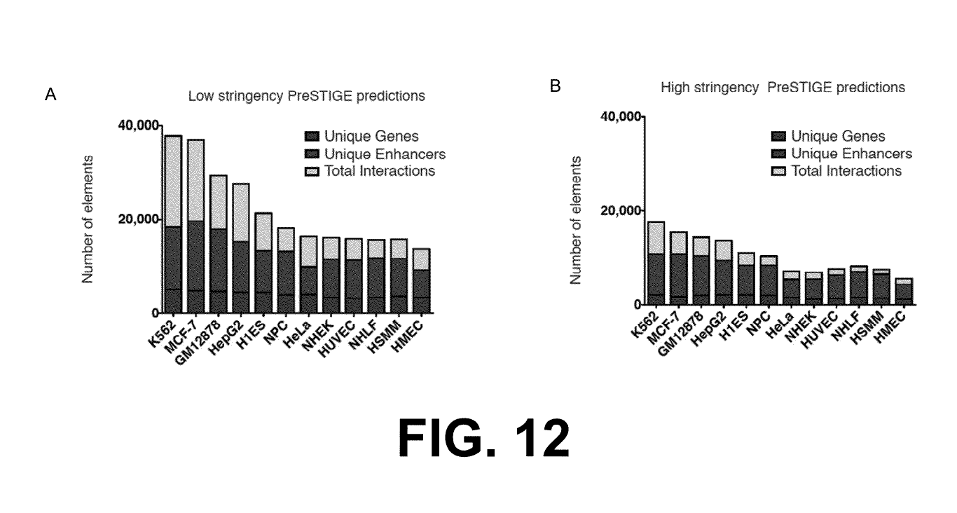

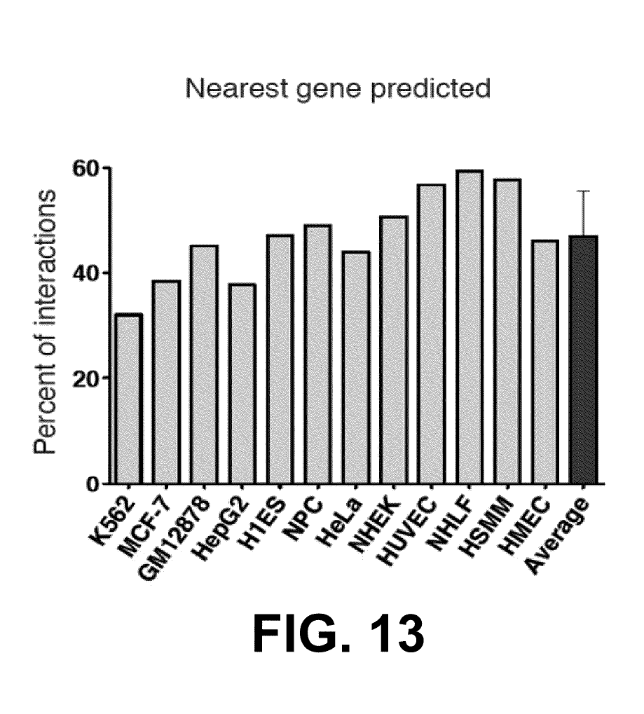

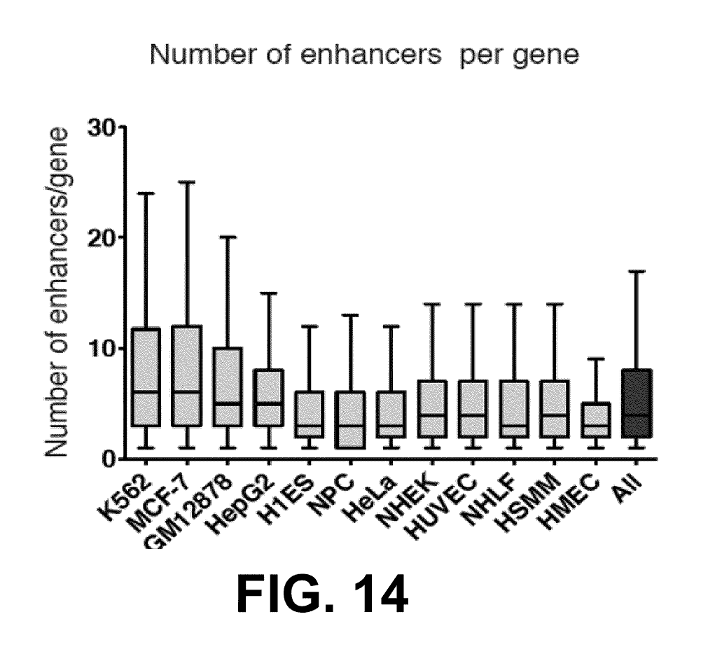

The number of total enhancer-gene predictions, unique genes, and unique enhancers determined at low and high stringency thresholds in each of the 12 cell types are shown in FIGS. 12A, 12B. Predictions include 28-46% of all expressed genes in each cell type. It was also determined that only 40% of PreSTIGE predictions involve an enhancer and the nearest gene (FIG. 13). Therefore, 60% of the PreSTIGE predictions would be undetected by nearest gene methods. Additionally the effect of the domain model on the predictions was evaluated. While most interactions occur within 100-kb, the use of CTCF sites in the domain model predicts enhancer-gene interactions that are separated by up to 5-Mb. These analyses also show that 42% of enhancer-gene interactions contained an intervening CTCF site that was “ignored” in the model. Less than 15% of PreSTIGE predicted interactions crossed topological domain boundaries, proposed to constrain gene-enhancer interactions. Lastly, predictions for individual genes and individual enhancers were analyzed. On average 5-6 enhancers were predicted to regulate a given gene (FIG. 14), although some genes were assigned to as many as 10-20 enhancers.

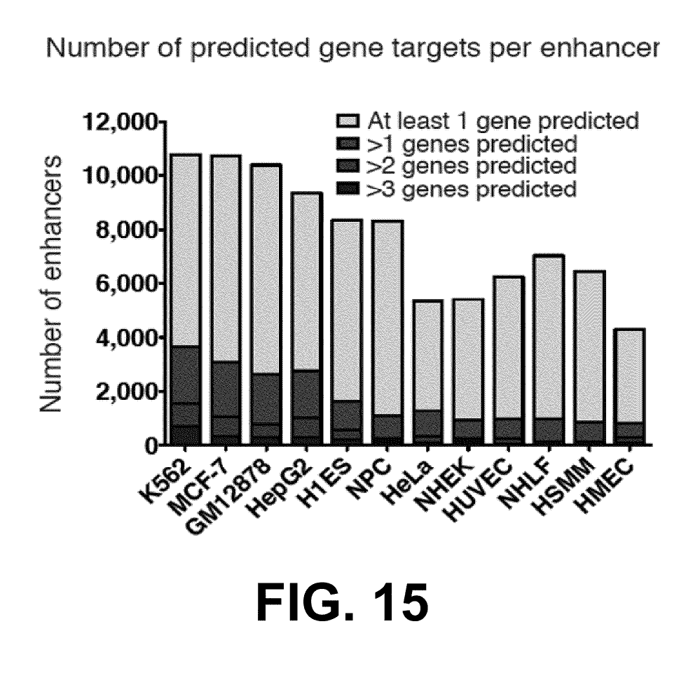

Genes associated with high numbers of enhancers were enriched for gene ontology terms “development” and “differentiation” (p-value <1E-10, by GREAT analysis, indicating that these classes of genes are under particularly exquisite regulatory control. The majority of enhancers were predicted to target only one gene. However, 22% of enhancers were predicted to regulate two or more genes, and 7% regulate three or more genes (FIG. 15). This suggests that interactions in which multiple genes share a common regulatory element, like the TH2 cytokine locus, may be more common than previously recognized. However, the possibility that this result is due to an enhancer that regulates one gene whose promoter is in physical contact with the promoter of a nearby gene cannot be excluded.

Annotation of SNPs Associated with Common Traits with PreSTIGE

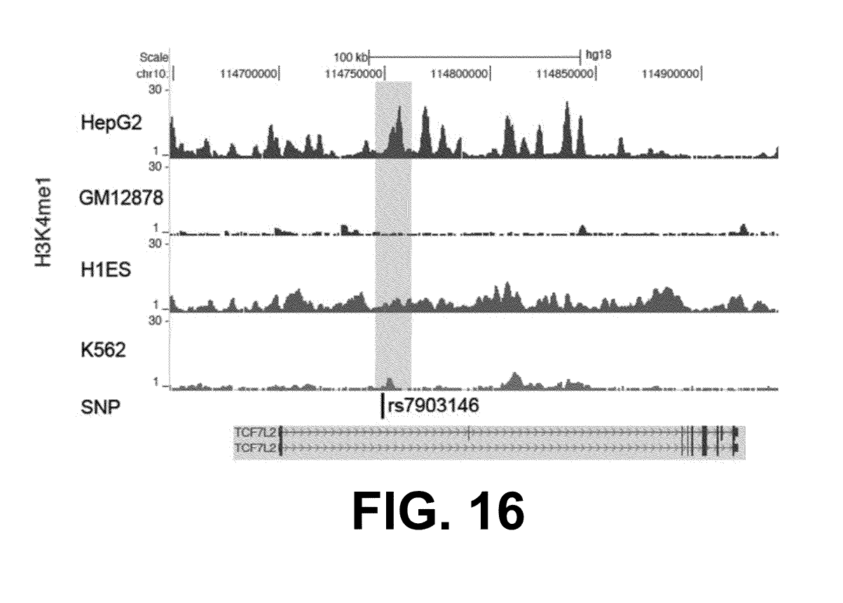

Having validated PreSTIGE and predicted enhancers for 28-46% of all expressed genes among 12 diverse cell types, the method was applied to GWAS. The entire NHGRI catalog of GWAS variants was downloaded, which as of September 2012 contained 7,106 SNPs associated with 627 traits. Because any SNP in linkage disequilibrium (LD) with the “lead” SNP may be responsible for the given trait, all SNPs in LD with each of the lead SNPs were retrieved. All GWAS entries for which the SNP or any of its LD SNPs fall within a coding region to identify 5,824 noncoding disease-associated SNPs were filtered out. LD and lead SNPs were then intersected with H3K4me1-enhancers in each of the 12 cell types in the reference set. PreSTIGE was next used to identify the gene targets of each enhancer containing a SNP. Remarkably, gene targets were predicted for 49.9% of all non-coding GWAS SNPs (2,904 out of 5,824), including predictions for 504 different traits. As an example, rs7903146, a type 2 diabetes SNP that maps to a HepG2 enhancer located within the intron of TCF7L2 (FIG. 16). PreSTIGE predicts this enhancer to regulate TCF7L2 expression and this prediction was validated by a previous study. The vast majority of the remaining 2,903 enhancer-SNPs target previously unidentified genes, of which >75% likely validate based on the FDR estimates. All 2,904 SNPs, as well as their associated enhancers, genes, and traits are listed in Table S2. Notably, of all predictions that PreSTIGE makes for GWAS variants, only 45.8% involve the nearest gene.

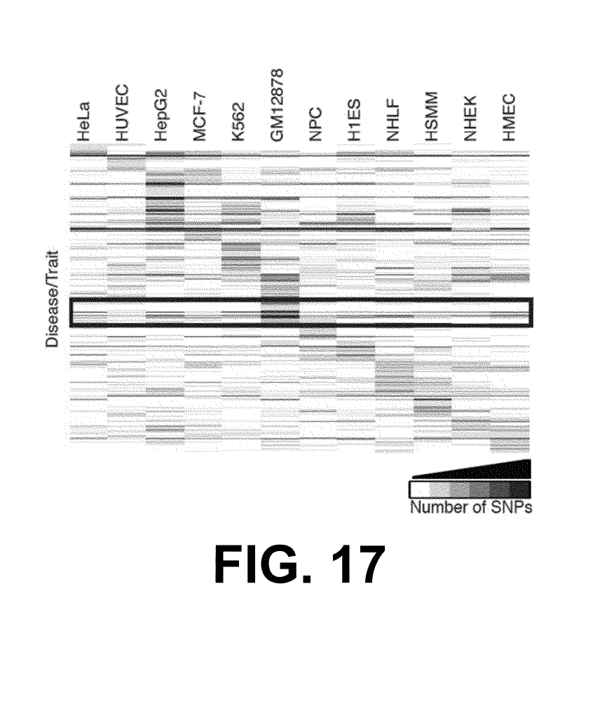

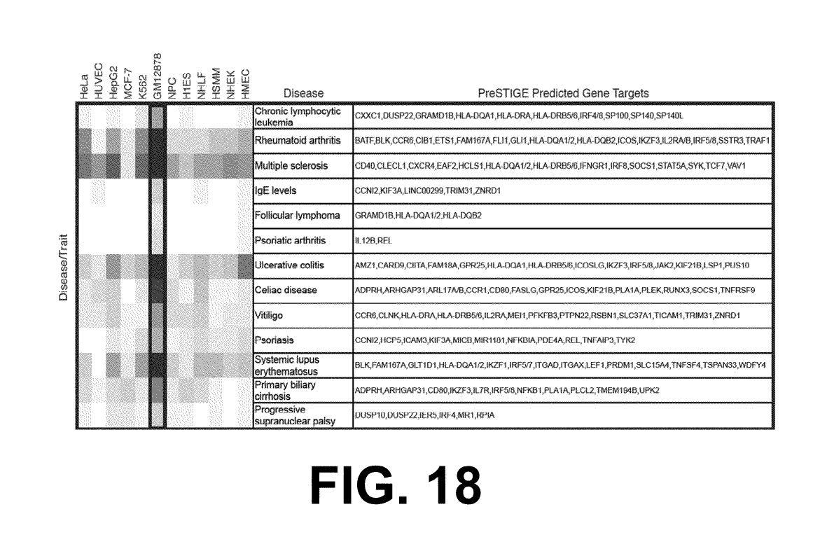

The number of GWAS SNPs that overlap enhancers of PreSTIGE predicted interactions were quantified in each cell type, hierarchically clustered the data, and plotted the results in a heatmap (FIG. 17). In accordance with previous observations, by and large, common disease SNPs correlate with enhancers in cell types generally considered relevant to the disease. Additionally, SNPs correlate with cell type-specific enhancer-gene interactions determined by PreSTIGE in cell types relevant to the disorder. For example, SNPs associated with brain-related traits such as autism, migraines, and Parkinson's Disease correlated with cell type-specific interactions in neural precursor cells. Immune-related disorders including rheumatoid arthritis, multiple sclerosis, Crohn's Disease and lupus correlated with PreSTIGE predicted interactions in cultured B lymphoblastoid cells (GM12878), derived from B cells (FIG. 18).

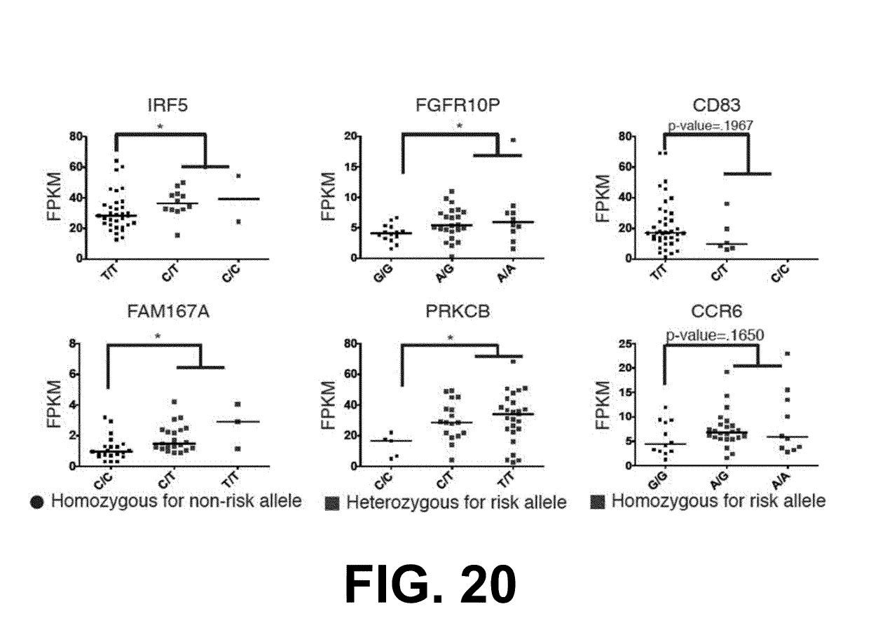

Variant Set Enrichment analysis was performed to test the statistical significance of the correlation between the immune-related risk SNPs and GM12878 (B lymphoblast) enhancers predicted by PreSTIGE. For comparison, the other 11-cell types and the colon crypt were also analyzed. Highly significant correlations were detected between risk SNPs associated with rheumatoid arthritis (RA) and Crohn's disease (CD) and B lymphoblast specific enhancers (FIGS. 20A, 20B). Other cell types showed correlations that were either insignificant or far less significant than those observed in the B lymphoblasts. These results suggest that B cell enhancers could be particularly relevant to the pathogenesis of these immune disorders.

Enhancer-SNPs that Predispose to Immune Disorders Impact Expression of their Target Genes

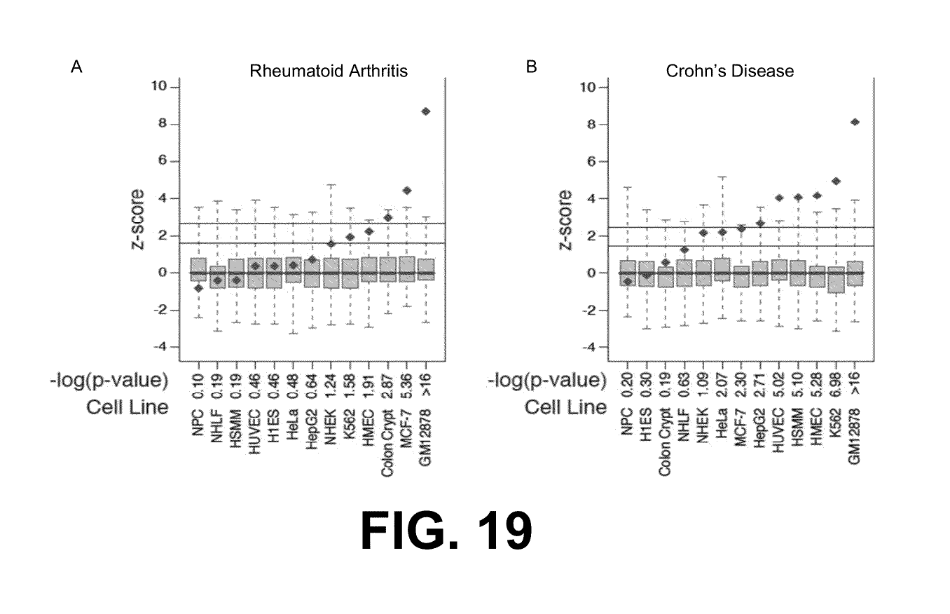

A key question is whether or not enhancer-SNPs associated with common traits influence expression of their gene targets. To address this question, findings indicating that B cell enhancers are significantly enriched among RA and CD-associated SNPs (FIGS. 19A, 19B), suggesting that B lymphoblasts can be exploited to study the effect of the immune disorder risk SNPs on gene expression. To this end, non-coding SNPs (and those in LD) associated with RA and CD were retrieved, and intersected with H3K4me1-enhancers predicted by PreSTIGE in GM12878 cells. In total, 19 RA SNPs and 17 CD SNPs (36 unique loci) overlapped a PreSTIGE predicted enhancer. Next, publicly available B lymphoblast expression data (RNAseq) from a panel of 61 CEU individuals was downloaded along with their corresponding genotypes. The 61 CEU individuals were then stratified by their genotypes (risk versus non-risk). After removing SNPs for which fewer than 4 individuals had the minor allele, 9 RA and 9 CD SNPs (18 unique loci) were left to evaluate transcript levels. 6/9 (66.7%) RA SNPs and 5/9 (55.6%) CD SNPs were associated with genes that showed differential expression between individuals carrying the risk and non-risk alleles (P<0.05). Results for six RA SNPs (4 significant and 2 insignificant) are shown in FIG. 20. By and large, the risk alleles were found to confer modest effects on target gene expression (<2.5 fold) and the effect was not always in the same direction. Specifically, of the 11 total SNPs found to significantly impact transcript levels, 6 elevated expression of the target gene, and 5 reduced expression.

### Common-Disease SNPs are Enriched in Enhancers of Highly Cell Type-Specific Transcripts

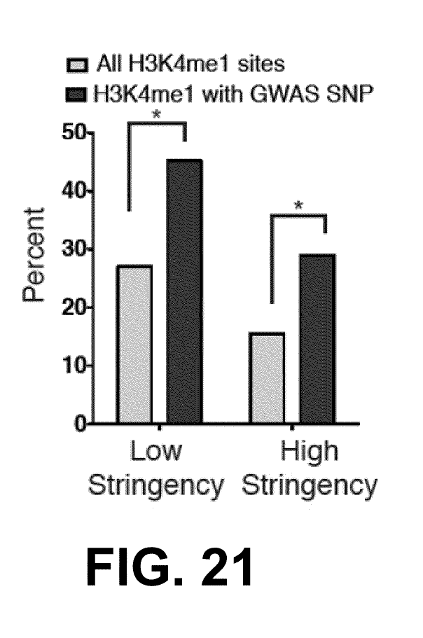

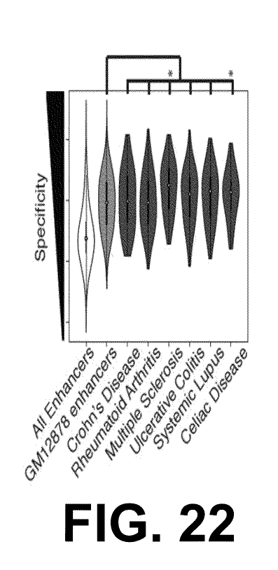

From the analysis above, it was noted that PreSTIGE identified gene targets of GWAS SNPs at a far higher rate than expected given PreSTIGE's baseline prediction rate. Overall, PreSTIGE identified gene targets for 45.1% of H3K4me1 sites that contain a GWAS SNP, while only 27% of all H3K4me1 sites were assigned a gene target (FIG. 21). Specifically in the B-lymphoblast cell type, 69.0% of RA-associated enhancer SNPs, and 52% of CD enhancer SNPs were assigned to gene targets. Given that PreSTIGE is biased toward analysis of cell type-specific enhancers and genes, this result could be explained if GWAS SNPs are more likely to map to cell type-specific enhancers, or if GWAS SNPs are more likely to regulate cell type-specific transcripts. To distinguish between these possibilities, the VSE analysis was expanded and to identify 4 additional traits significantly associated with B-lymphoblast enhancers: multiple sclerosis, ulcerative colitis, systemic lupus and celiac disease. Each enhancer was then “scored” by its relative level of specificity in GM12878 cells compared to the 11 other cell types. Specificity scores for all GM12878 enhancers were then compared to the specificity of those containing GWAS SNPs. By and large, the overall range of specificity scores was comparable between all GM12878 enhancers and those containing GWAS SNPs (FIG. 22, compare dark gray to lighter gray). The relative specificities of predicted transcripts were then compared. GM12878 predicted transcripts showed a lower range of specificity than predicted transcripts associated with disease variants for all 6 traits analyzed (FIG. 4F, compare red to grey). Thus, the correlations between GWAS SNPS, enhancers, and PreSTIGE predictions are not only driven by the enrichment of GWAS SNPs in cell type-specific enhancers, but also by GWAS SNPs regulating highly cell type-specific transcripts. These findings indicate that GWAS enhancer variants fall in a unique class.

### Common Disease SNPs Often Involve Multiple Enhancer Variants in LD

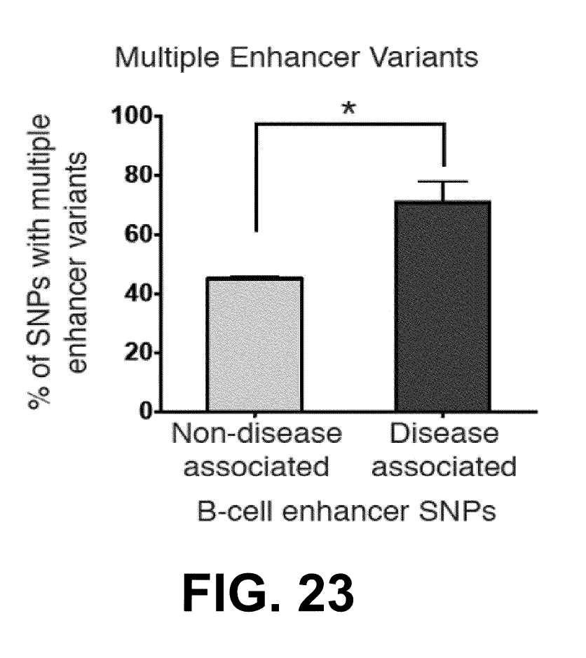

On average PreSTIGE assigned 4-5 enhancers per gene. These results prompted a test of how often a GWAS enhancer SNP predicted to regulate a given gene is in LD with at least one other variant in a different enhancer that is predicted to target the same gene. The six traits significantly associated with Blymphoblast enhancers were utilized through VSE analysis (RA, CD, MS, ulcerative colitis, celiac disease, and systemic lupus). On average, 70.7% of enhancer variants were in LD with SNPs that fall in an additional predicted enhancer of the same gene. By comparison, only 45% of SNPs that fall within B-cell specific enhancers that are not associated with a disease involve multiple enhancer variants in LD with one another (P<0.0001, FIG. 23). These suggest that for many common traits, there is not a single disease-causing variant that underlies the association signal, but rather several distributed among multiple enhancers of a given gene (the “multiple enhancer variant” hypothesis).

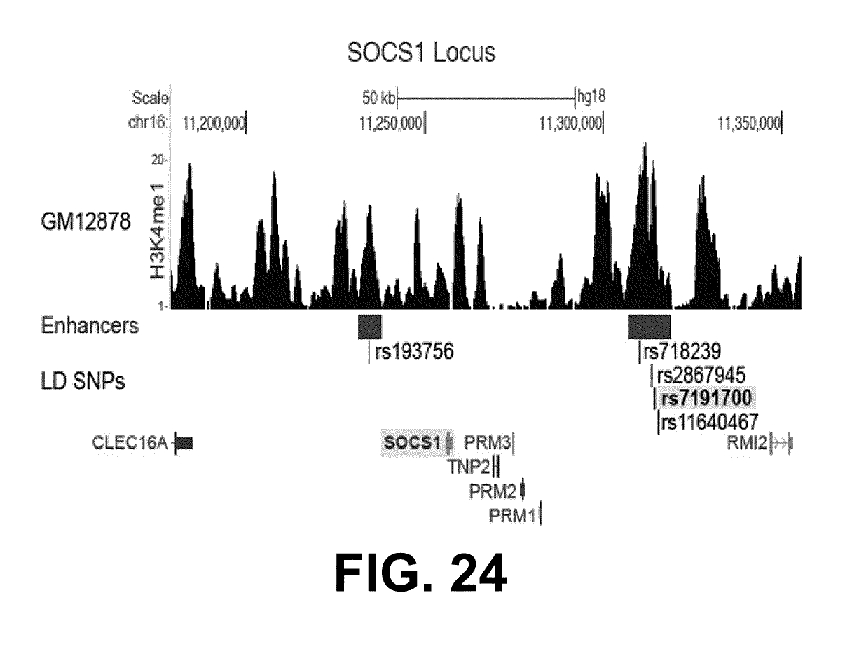

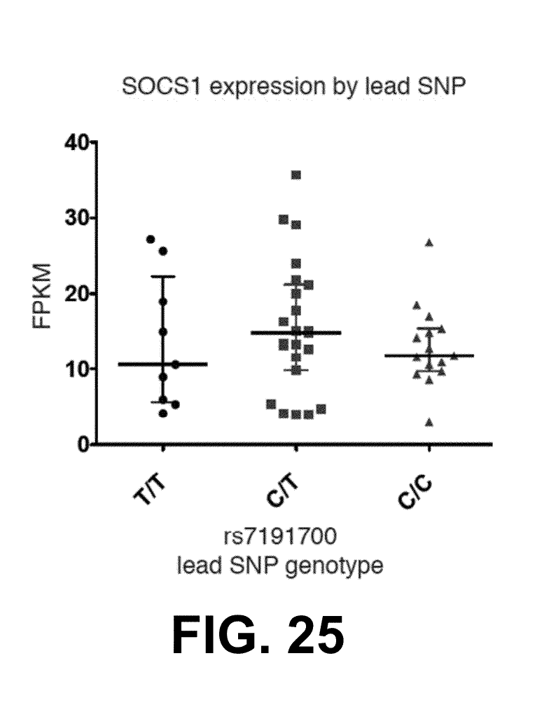

To test this hypothesis available lymphoblastoid expression data (RNA-seq) was matched with genotype from CEU individuals. When multiple enhancer variants are in perfect LD at a given locus it is difficult to separate the effect of each individual enhancer variant on target transcript levels, because the variants always travel together. Regions of ‘imperfect LD’ were identified. In these regions the SNPs are reported to be in LD (see methods) among the CEU population, but there are several individuals within the 60-person panel in which the LD block structure is split in such a way that enhancer variants are not necessarily inherited together. These loci provide an opportunity to evaluate whether more than 1 enhancer variant alters target transcript levels. An example is shown in FIG. 24. Here, a risk SNP for Multiple Sclerosis (rs7191700) maps to the SOCS1 enhancer in GM12878 cells. Four additional SNPs (rs718239, rs2867945, rs7191700, and rs11640467) are in LD with rs7191700 and map to an additional enhancer of SOCS1. Individuals were subdivided into three groups based on their genotype at the multiple sclerosis GWAS SNP rs7191700: (1) those homozygous for the non-risk allele (C/C), (2) those heterozygous for the risk allele (C/T), and (3) those homozygous for the risk allele (T/T). SOCS1 transcript levels were plotted, and no significant differences in SOCS1 levels were observed between individuals with the non-risk allele and those with the risk allele FIG. 25. Similar results were observed for all four remaining SNPs in LD with the lead SNP rs719700 (not shown). The genotype for the four SNPs in LD with the lead SNP rs719700 were determined and the individuals stratified based on their haplotype, considering phase.

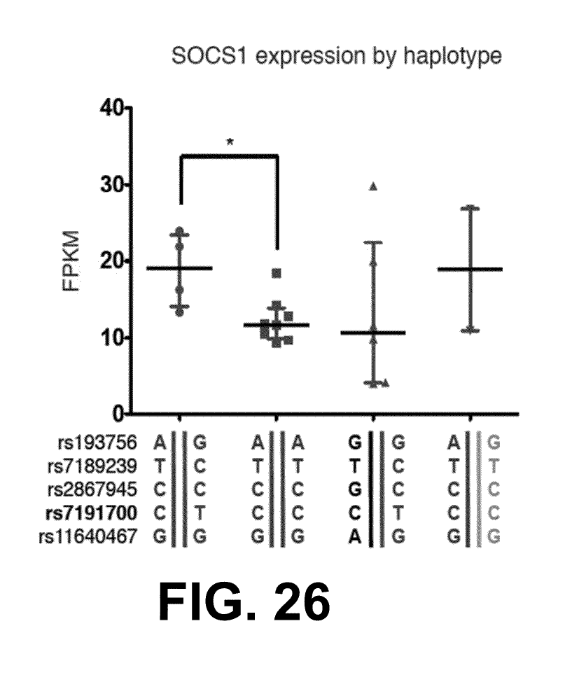

Remarkably, when the genotype of the lead SNP was considered along with the four LD SNPs located in the other SOCS1 enhancer, the effect of the risk haplotype on SOCS1 levels was clearly evident (FIG. 26). Thus, the effect on gene expression depends on the genetic makeup of the whole haplotype, not just one enhancer SNP. Moreover, the SNPs associated with both enhancers need to be considered when assessing the influence on gene expression. In total, 5 GWAS loci similar to SOCS1 were identified, where there was no difference in expression based the genotype of any of the individual enhancer variants in LD, but a difference in gene expression is seen when the entire haplotype was considered.

### Example 2

This example shows that PreSTIGE can be applied to different species (e.g., a mouse with PreSTIGEouse).

### Methods

Predicting Gene Targets of Enhancers with PreSTIGE

The PreSTIGE bioinformatics algorithm was utilized to associate enhancer-gene pairs. PreSTIGE predicts enhancer-gene pairs based on genomic proximity (<100 kb) and shared specificity of enhancer H3K4me signal and gene expression as compared to a panel of 12 tissues. PreSTIGE was initially developed to interrogate human enhancer-gene pairs and was adapted for application to mouse (PreSTIGEouse).

### Results

### Enhancer Profiles Distinguish Mouse Pluripotent Stem Cells

To understand the differences in transcriptional regulation between mESCs and mEpiSCs, epigeonomic and transcriptome profiling of these two pluripotent cell types using high-quality ChIP-seq and RNA-seq data sets. The epigenomic analysis was focused on cis-regulatory regions known to be marked by specific chromatin features:H3K4me1, associated with putative enhancer elements; H3K4me3, associated with transcription start sites (TSSs); H3K27ac, enriched at active promoters and enhancers; H3K27me3, generally associated with transcriptionally repressed regions of chromatin; and DNase-seq, indicative of open regions of chromatin.

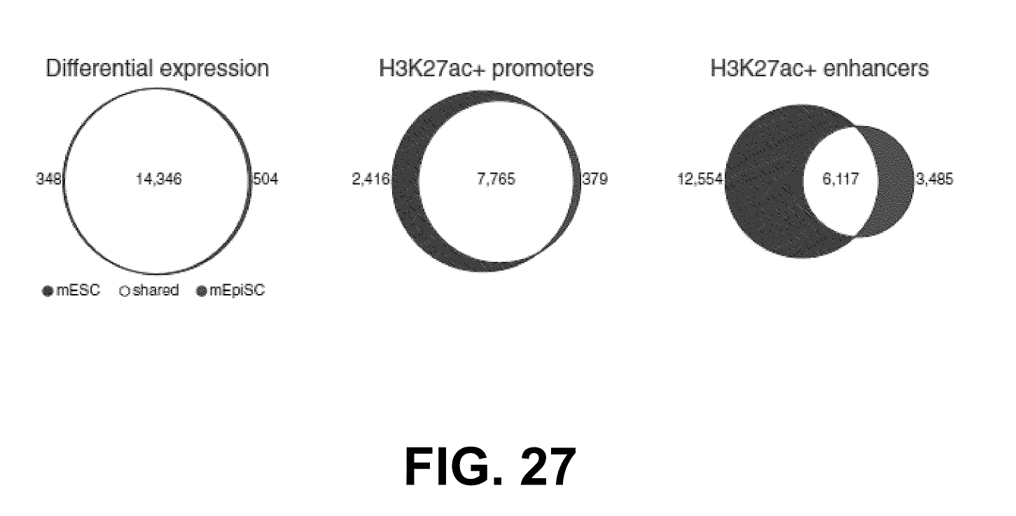

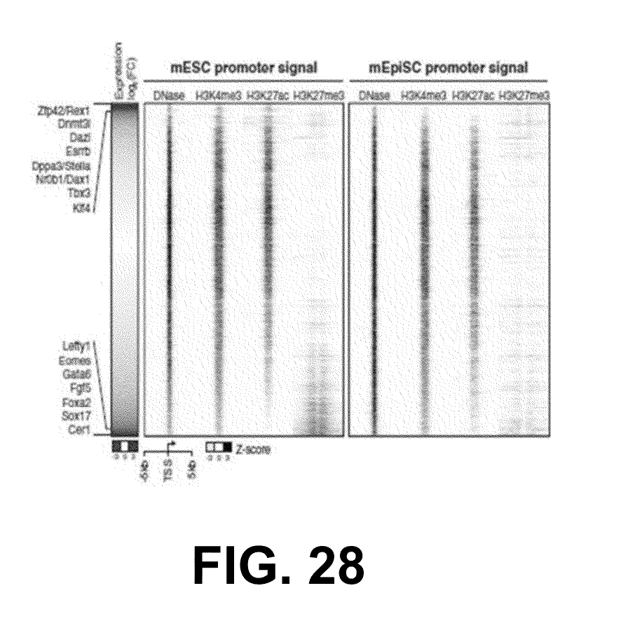

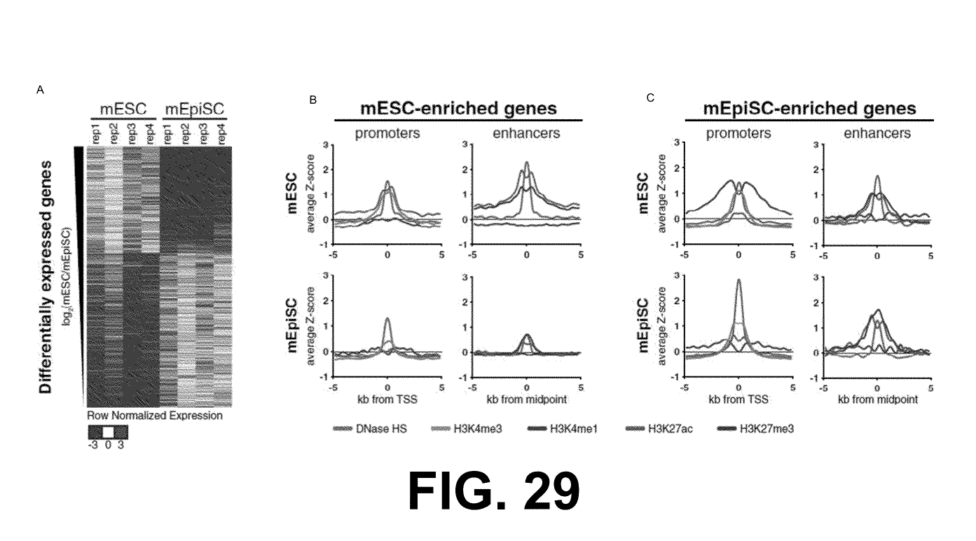

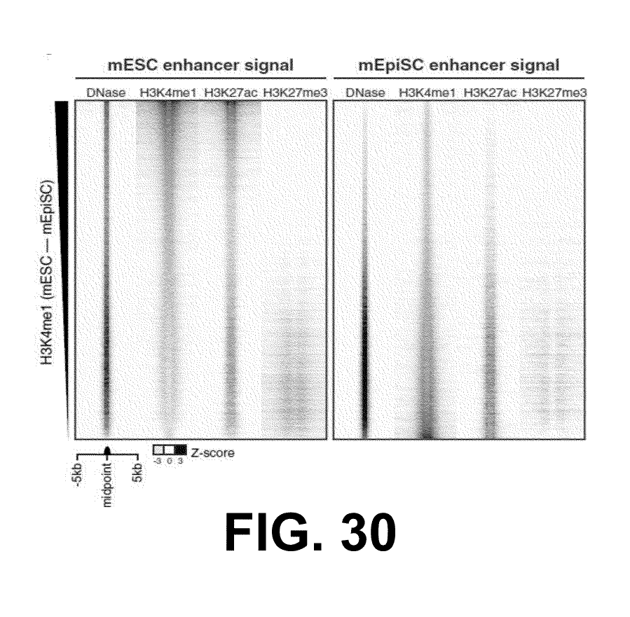

Through analysis of the RNA-seq and ChIP-seq data, the number of transcripts differentially expressed between mESCs and mEpiSCs, as well as the number of promoters and enhancers that showed chromatin state differences between the two cell types were determined (FIG. 27). The transcriptomes of mESCs and mEpiSCs were remarkably similar to one another (R2=0.83; n=4 biological replicates per cell type), with less than 6% (852 out of 15,198) of expressed transcripts (fragments per kilobase per million mapped reads [FPKM]>0.25) showing a significant difference in abundance between mESCs and mEpiSCs. Among this list were transcripts of genes known to be mESC specific, including Esrrb, Zfp42, Dppa3, and Klf4, as well as those known to be mEpiSC specific, including Fgf5, Cer1, and Lefty1 (FIG. 28). Global promoter states were also largely similar, with over 73% of 10,560 active (H3K27ac+) promoters overlapping between the two cell types (FIGS. 27 and 28). Even promoters of differentially expressed genes (referred to as mESC-enriched and mEpiSC-enriched) showed a similar state of DNase hypersensitive open chromatin (FIGS. 29A, 29B, 29C). In stark contrast, chromatin states at the 22,156 H3K4me1+ H3K27ac+ enhancer loci were much more distinctive, showing only 27% overlap (FIGS. 27 and 30). These data suggest that the enhancer landscape may play a dominant role in defining differences between pluripotent cell types on a molecular level.

The computational approach called PreSTIGEouse was used to assign enhancers to mESC-enriched and mEpiSC-enriched genes. Enhancers of mESC enriched genes contained all signature features of active elements in mESCs (H3K4me1, H3K27ac, and DNase hypersensitivity) and were decommissioned in mEpiSCs (FIG. 29B). Globally, enhancers of mEpiSC-enriched genes are marked by an active enhancer signature in both cell types (FIG. 29C). Half of the enhancers of mEpiSC-enriched genes lack marks of active enhancer elements in mESCs, while half are marked by an active enhancer signature in both. These data suggest that the enhancer landscape of mESCs is primed to activate mEpiSC-enriched genes at the successive developmental stage. However, fewer than 8% of enhancers that differ between mESCs and mEpiSCs could be accounted for by differences at mESC- and mEpiSC-enriched genes.

Pluripotency-Enriched Genes Show Dramatic Enhancer Differences between Pluripotent States

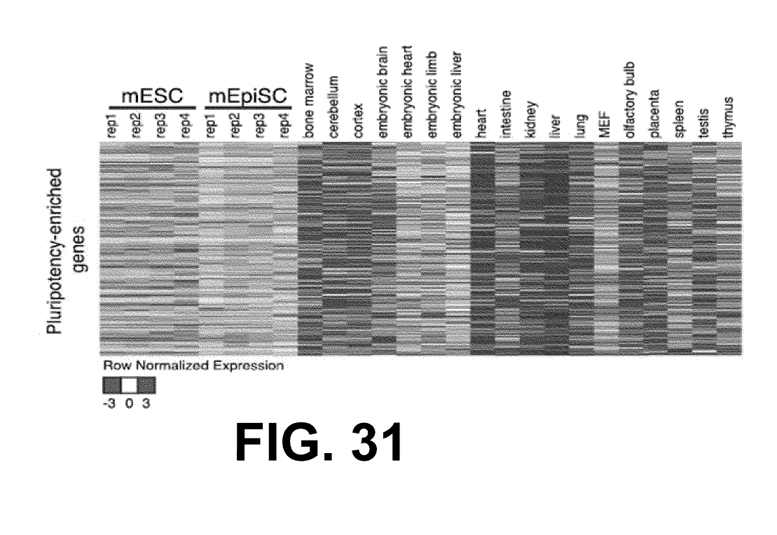

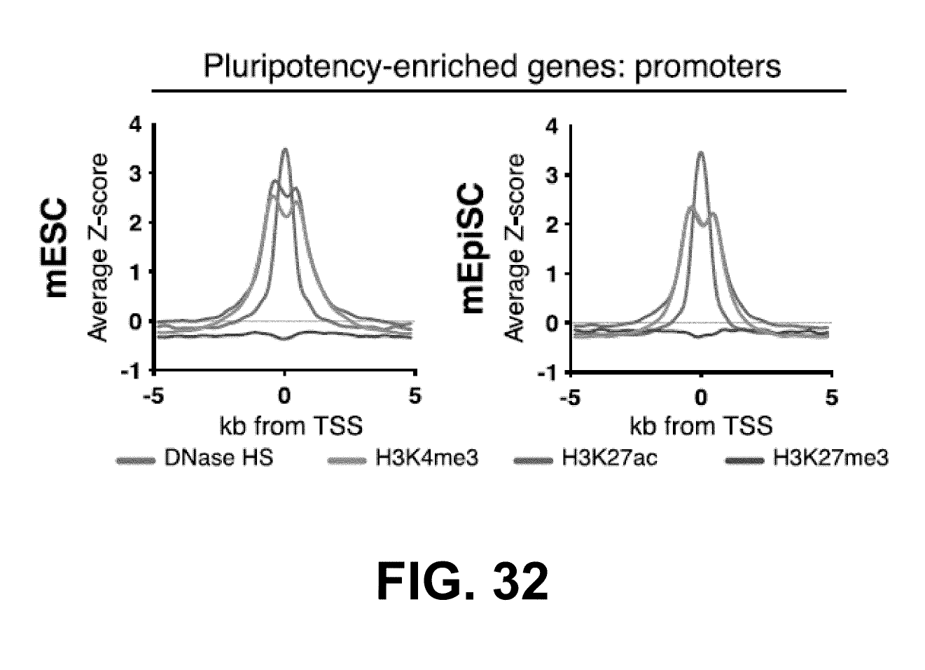

To further explore the global enhancer differences between mESCs and mEpiSCs, it was explored whether genes expressed at similar levels between mESCs and mEpiSCs, including many pluripotency-related factors, are regulated by distinct enhancers. It is known that the Pou5f1, also known as Oct3/4, locus is controlled by distinct enhancers in the preimplantation inner cell mass versus the postimplantation epiblast in vivo as well as in mESCs versus mEpiSCs in vitro. However, it was not known whether this represents a global regulatory phenomenon. To test this hypothesis, a set of “pluripotency-enriched” genes was defined using RNA-seq data sets from mESC, mEpiSCs, and a panel of 18 developmental and adult mouse cell and tissue types. Stringent metrics were set to ensure that genes within this class are expressed at similar levels in mESCs and mEpiSCs (p>0.05 and <2-fold change between mESCs and mEpiSCs) and enriched in the two pluripotent cell types in comparison to a panel of 18 different mouse tissues (FIG. 31). As expected, this pluripotency-enriched gene class contained gene ontology terms consistent with pluripotent phenotypes and the genes share a nearly identical active chromatin state at their promoters (FIG. 32). Consistent with their role in regulating key cell identity genes, 25% of these pluripotency-enriched genes were associated with super enhancers in either of the two cell types. However, the locations of these super enhancers in the two cell types were largely nonoverlapping, indicating a distinct control mechanism even for these similarly expressed pluripotency-enriched genes.

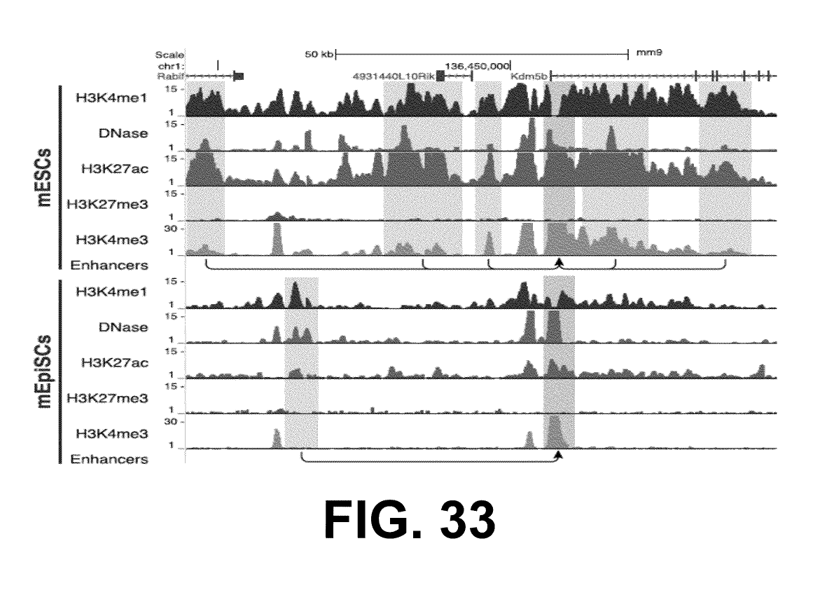

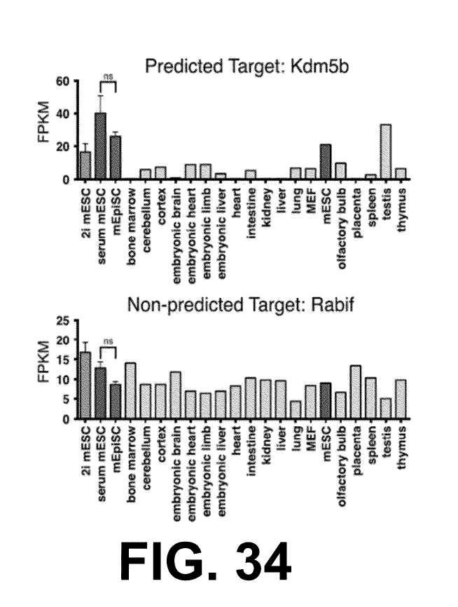

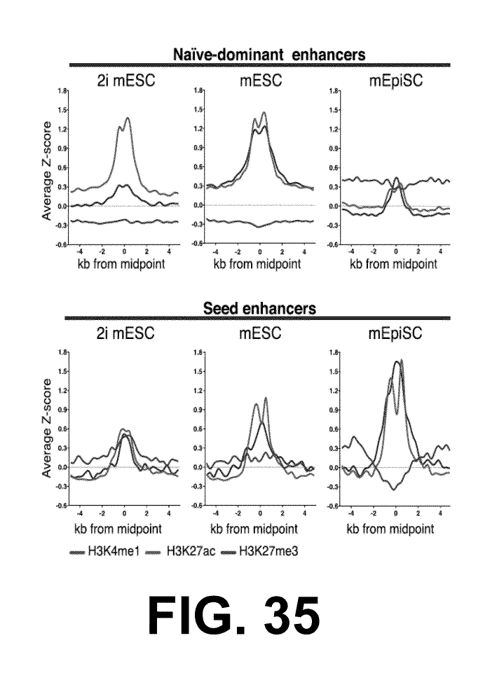

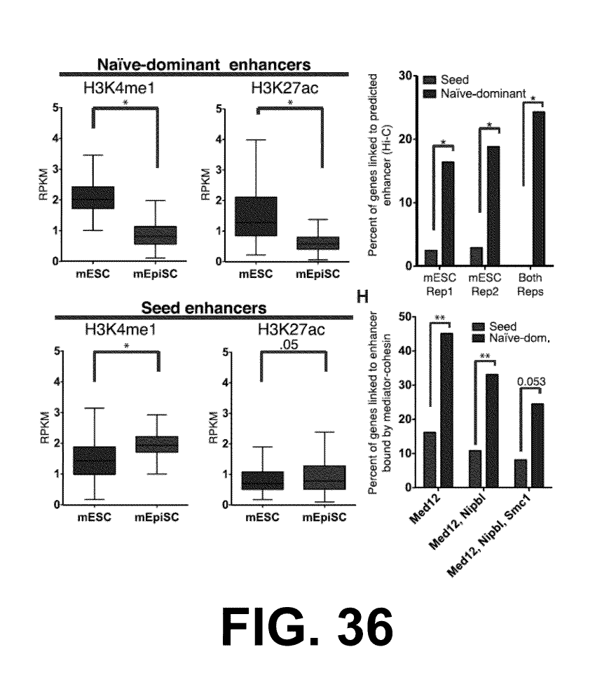

Enhancer elements were assigned to each pluripotency-enriched gene using a computational approach called PreSTIGEouse. Of the 602 pluripotency-enriched genes, 97% showed evidence of differential enhancer usage between the two pluripotent cell types. An example is shown in FIG. 33. Here, the five enhancer elements predicted in mESCs to regulate Kdm5b, an exemplar pluripotency-enriched gene, are highlighted in blue (FIGS. 33 and 34). All five enhancers are located in open chromatin and contain high levels of H3K4me1 and H3K27ac (FIG. 33). In mEpiSCs these five enhancers are virtually devoid of H3K4me1 and H3K27ac, and a different enhancer (highlighted in red) is predicted to regulate Kdm5b. All enhancers that, like those associated with Kdm5b in mESCs, were predicted to regulate pluripotency-enriched genes exclusively in mESCs. These enhancers, called “naive-dominant,” generally contained high levels of H3K4me1 and H3K27ac in mESCs relative to mEpiSCs, where enhancer-histone signals were low or near background levels (FIGS. 35 and 36). Thus, most naive-dominant enhancers are largely inactivated, or “lost” upon transition of mESCs to the mEpiSC state. Next, “primed-dominant” enhancers, or those predicted to regulate the pluripotency-enriched genes exclusively in mEpiSCs, were selected. As expected, these enhancers contained high levels of the active enhancer marks H3K4me1 and H3K27ac in mEpiSCs (FIGS. 35 and 36). However, unexpectedly, these same enhancers were enriched for H3K4me1 and H3K27ac in mESCs, albeit at lower levels than in mEpiSCs (FIGS. 35 and 36). These enhancers were not assigned to gene targets in mESCs due to the relatively low levels of the associated enhancer histone marks. As a result of their apparent switch from dormancy in the naive state to active transcriptional regulation in the primed state, referred to as “seed enhancers.” Seed enhancers were compared to a class of “shared enhancers,” i.e., enhancers predicted to regulate pluripotency-enriched genes in both cell types. Despite their relatively low signal intensity in mESCs, seed enhancers contain the highest levels of H3K4me1 and H3K27ac among all mEpiSC enhancers of pluripotency-enriched genes, suggesting the importance of the seed enhancer in the active regulation of the pluripotency-enriched genes.

To test whether the chromatin signal at seed enhancers in mESCs could be an artifact caused by metastable heterogeneity within mESC cultures, it was tested if the seed enhancers were present in mESCs grown under defined “2i” culture conditions, which are thought to promote a more homogeneous population of naive cells. It was found that, similar to mESCs grown in standard conditions, nearly all seed enhancer loci are marked by H3K4me1 in mESCs grown in 2i conditions and that half of these sites are also marked with H3K27ac, while the other half are marked with H3K27me3 (FIG. 35). Additionally, the levels of Pecam1 expression can distinguish mESCs in culture that are more naive-like from cells that are more epiblast-like. FACS was used to separate mESCs into Pecam1-high and Pecam1-low populations, followed by immediate fixation and ChIP-seq. The seed enhancers were found to be present at nearly identical levels in both populations, further supporting the idea that the presence of seed enhancers in mESCs is not the result of a contaminating epiblast-like population.

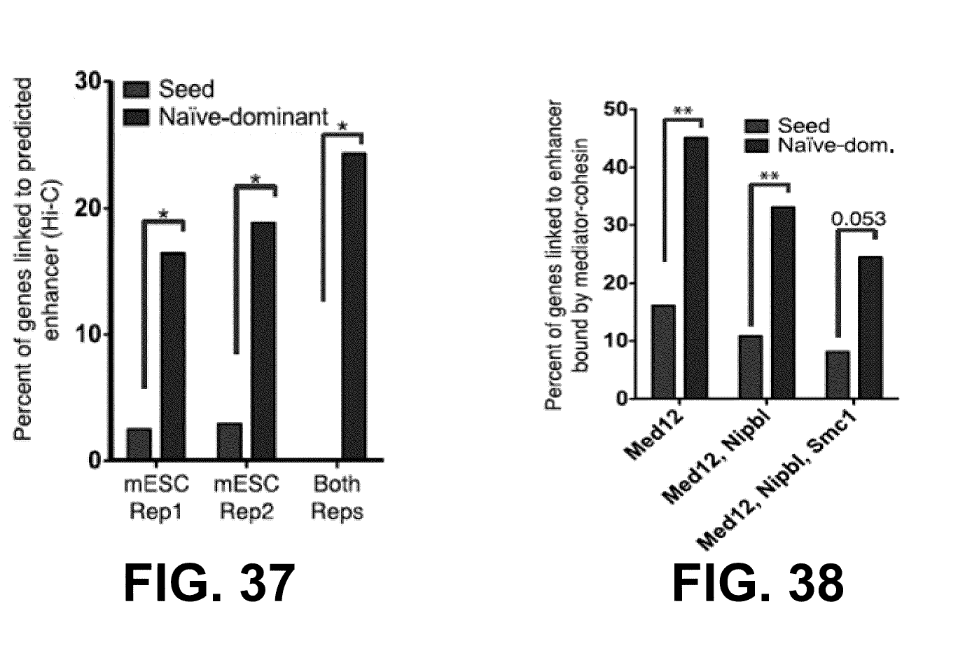

Using publically available chromatin interaction maps generated through Hi-C experiments, the fact that compared to naive-dominant enhancers, seed enhancers rarely physically interact with the promoters of pluripotency-enriched genes in mESCs was validated (FIG. 37). Additionally, compared to naive dominant enhancers, seed enhancers are in frequently occupied by components of the mediator-cohesion complex (Med12, Nipb1, and Smc1) in mESCs, which are known to physically link enhancers with their target promoters (FIG. 38). Seed enhancers show increased sequence conservation and are generally located farther from the TSSs of genes they control than naive-dominant enhancers. Motif analysis of the two classes revealed that many of the transcription factors enriched at seed enhancers overlap with those found at naive-dominant enhancers. It is interesting that naive-dominant enhancers exclusively are enriched for both known naive-specific factors, such as Esrrb and Tcfcp211, and pluripotency factors, such as Oct4, Nanog, and Sox2. Additionally, naive-dominant enhancers are enriched for motifs of Smad2/3 and Smad4, key downstream mediators of the Activin/Nodal pathway required for mEpiSC maintenance. Collectively, these results suggest a mechanism by which Activin/Nodal signaling may be required for repression of naive-dominant enhancers during the transition to primed pluripotency.

It was asked if the control of pluripotency-enriched genes by seed enhancers was a mouse specific molecular feature or if it extended more broadly to other mammalian pluripotent cells such as hESCs. Although embryo-derived hESCs exist in a primed pluripotent state similar to mEpiSCs, recent work has shown that hESCs can be converted into a naive-like state using extrinsic factors. Available enhancer-histone modification ChIP-seq data from this model system were used to test if the dynamic enhancer changes observed in mESCs and mEpiSCs are recapitulated in human pluripotent stem cells. To do this, a set of human pluripotency-enriched genes were selected and assigned to enhancers using methods similar to those used in mouse. Similar to observations in mouse cells, these human cells contained robust naive-dominant enhancers that lack chromatin markers of enhancer activity in the primed state, as well as seed-like enhancers with more robust activity in the primed state than the naive cell state. These data suggest that an early developmental transition from a naïve pluripotency-reinforcing epigenomic state to a primed somatic differentiation-capable state may be a general phenomenon of mammalian development.

Seed Enhancers are Utilized in Downstream Tissues Where They Expand into Enhancer Clusters

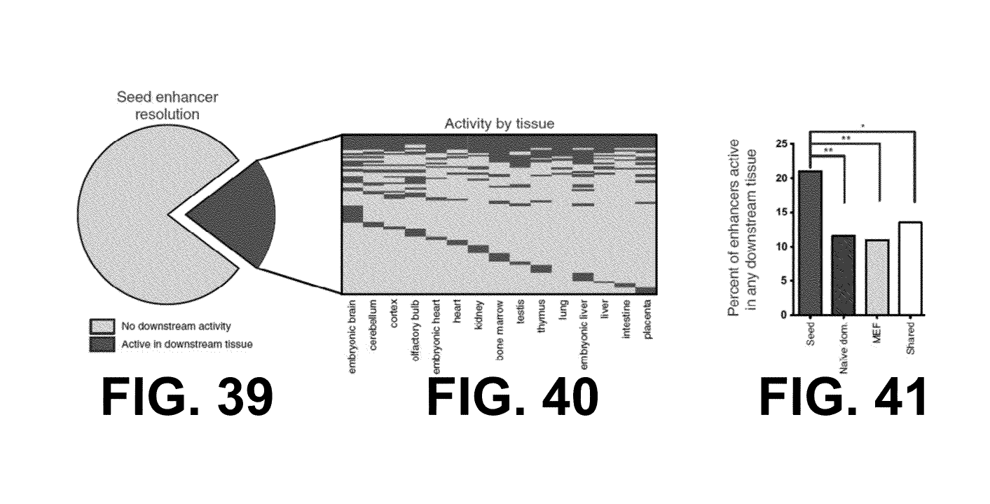

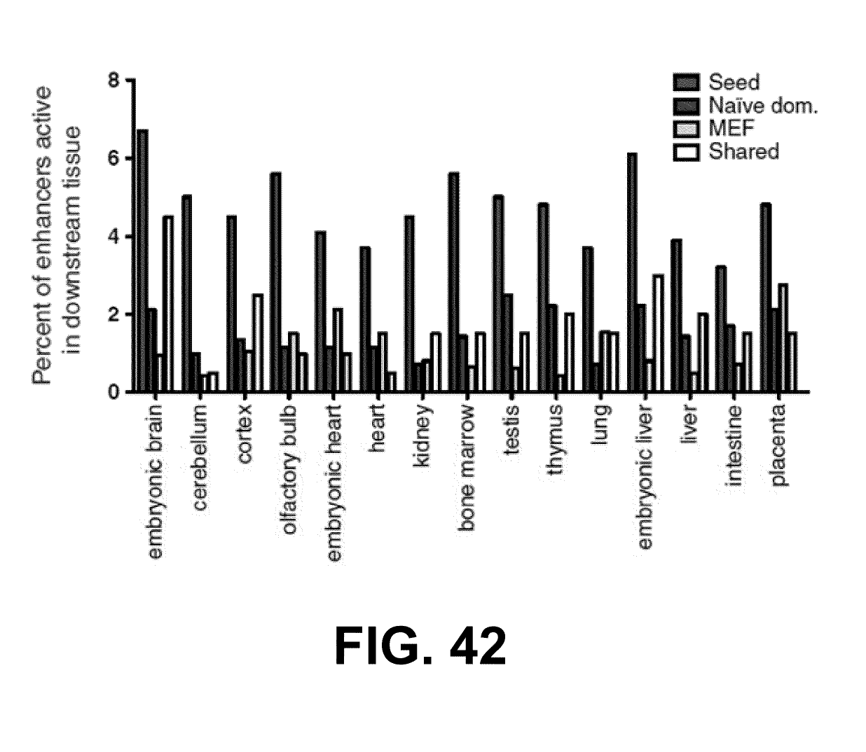

Collectively, the findings suggest that the global transcriptional control of pluripotency genes is quite distinct between the naïve and primed phases of pluripotency. While genes have been shown to be controlled by distinct enhancers in completely different cell types, it was puzzling as to why genes expressed at virtually identical levels in successive cell stages would undergo enhancer switching. This question prompted an investigation of additional features of seed enhancers. Given that seed enhancers show greater sequence conservation than typical enhancers, they might play a role at later stages of development. Using available H3K27ac ChIP-seq data from 15 different mouse embryonic and adult tissues, it was found that 21% of seed enhancers were significantly enriched for H3K27ac in at least one tissue (FIGS. 39 and 40). The rate of seed enhancer usage in downstream tissues was nearly double that of naive-dominant enhancers shared enhancers, and a control set of mouse embryonic fibroblast enhancers (indicative of the background rate of enhancer usage in multiple tissues) (FIG. 41). This trend held true across all 15 tissues (FIG. 42).

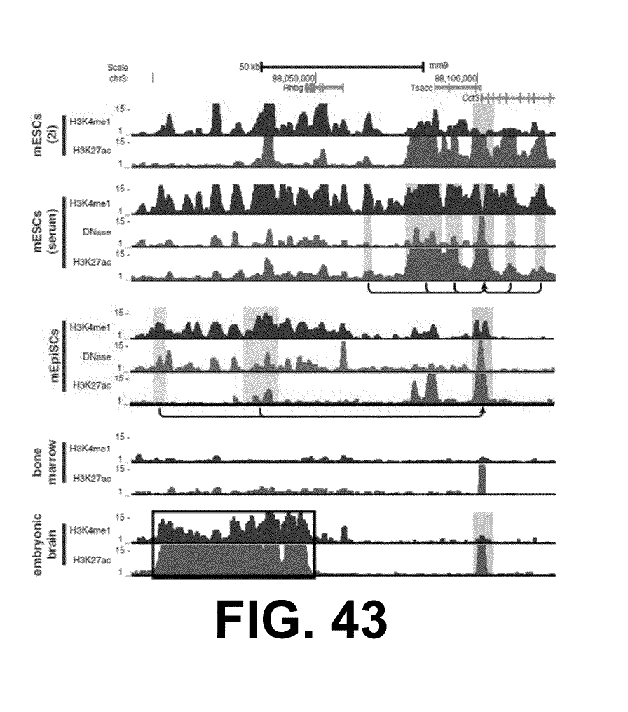

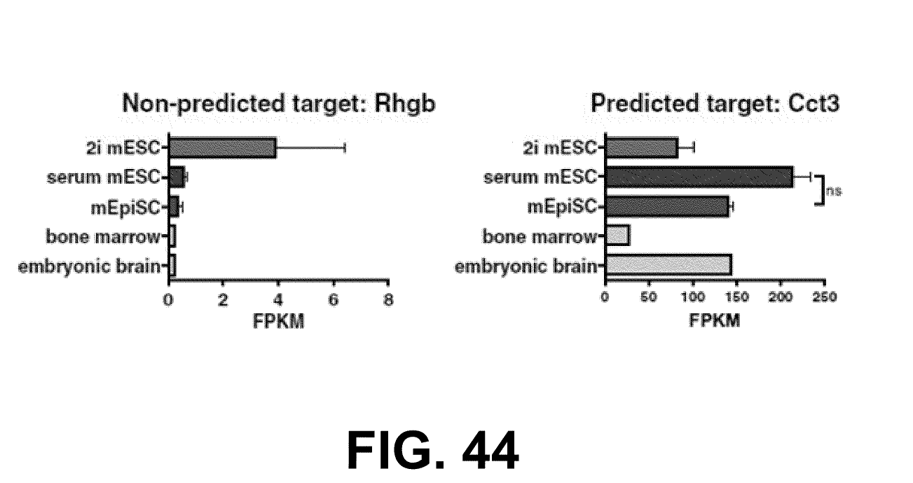

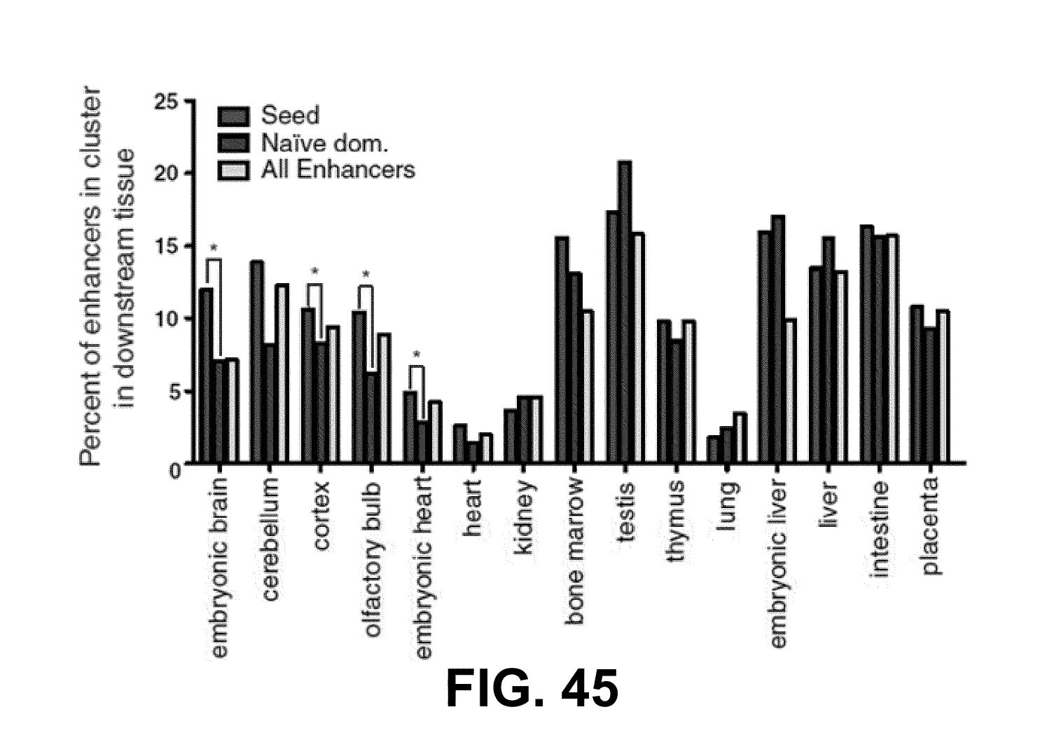

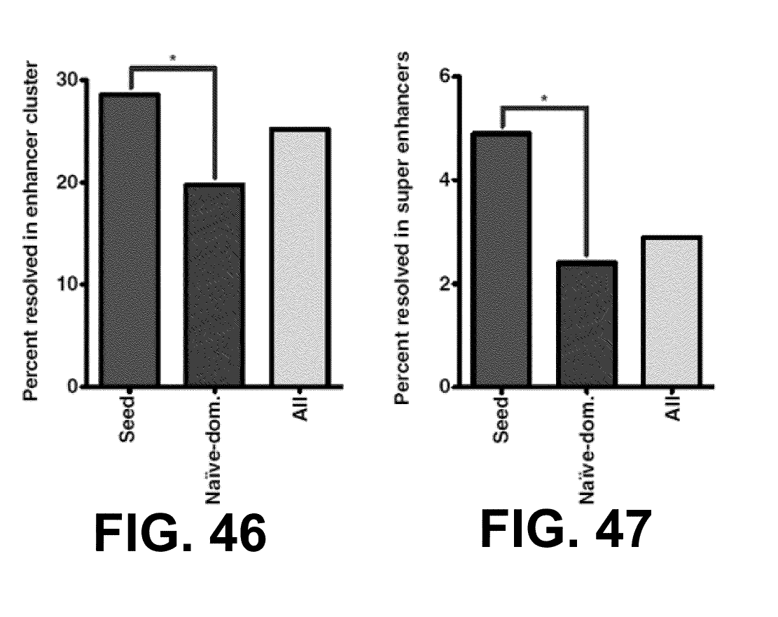

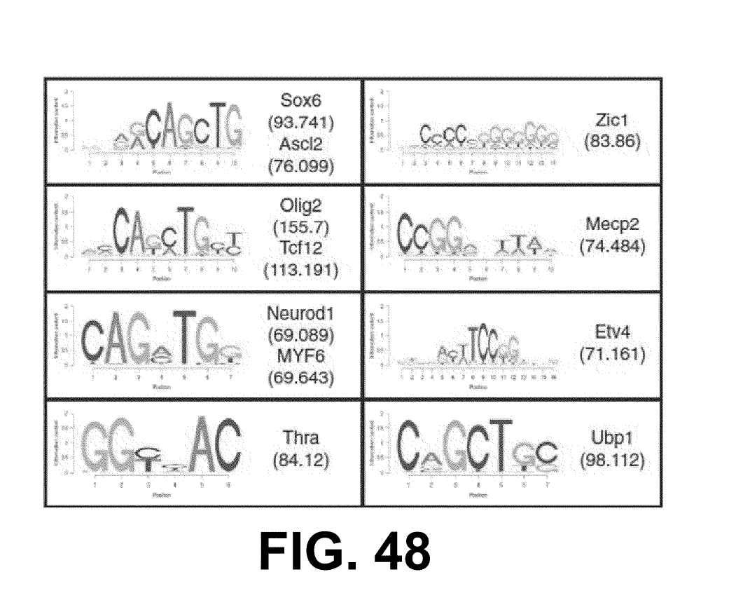

Upon visual inspection of the seed enhancers in the downstream tissues, several were contained within large domains of chromatin broadly marked with H3K4me1. For example, the Cct3 gene is a pluripotency-enriched gene regulated by two seed enhancers in mEpiSCs (FIG. 43) that is also expressed in embryonic brain (FIG. 44). Both seed enhancers become components of a large enhancer domain in embryonic brain. Domains like these are likely composed of multiple individual enhancer elements and are reminiscent of super and stretch enhancers. To test the significance of these observations, it was determined that the number of seed enhancers that lie within an enhancer cluster (defined as four or more enhancers within 100 kb) in a downstream tissue (FIG. 45). Seed enhancers were more likely than naive-dominant enhancers to lie within enhancer clusters in four different tissues: embryonic brain, cortex, olfactory bulb, and embryonic heart (FIG. 45). By comparison, naive-dominant enhancers were not significantly enriched in enhancer clusters of any downstream tissue tested. Given the propensity for seed enhancers to lie within neural related enhancer clusters, how often a seed enhancer gives rise to an enhancer cluster in at least one of the four neural tissues in the panel (embryonic brain, cortex, olfactory bulb, and cerebellum) were identified and it was found that 29% of all seed enhancers fall within an enhancer cluster in one of these neural tissues (FIG. 46). The same enrichment was found for seed enhancers in regions that become super enhancers in these four neural tissues (FIG. 47). Interestingly, given the tendency for seed enhancers to contribute to super enhancers or enhancer clusters in neural tissues, seed enhancers that resolve in any downstream tissue are enriched for motifs associated with neural lineage transcription factors, including Asc12, Sox6, Olig2, and Neurod1 (FIG. 48).

The aspects of this disclosure have been described illustratively. Accordingly, the terminology employed throughout the disclosure should be read in an exemplary rather than a limiting manner. Although minor modifications of the invention will occur to those well versed in the art, it shall be understood that what is intended to be circumscribed within the scope of the patent warranted hereon are all such embodiments that reasonably fall within the scope of the advancement to the art hereby contributed, and that that scope shall not be restricted.

What is claimed is:

1. A system, comprising:
a non-transitory memory that stores machine-readable instructions; and
a processor configured to access the memory and execute the machine-readable instructions;
wherein the machine-readable instructions comprise:
a gene selector that receives an input of a gene from a given cell type;
a domain selector that identifies an enhancer located within a domain established across the gene; and
a correlater that tests a pair of the enhancer and the gene based on a comparison to a set of known enhancer-gene pairs.

2. The system of claim 1, wherein the correlater predicts whether the enhancer targets the gene based on the comparison.

3. The system of claim 1, wherein the domain selector selects a preliminary domain comprising at least 100 kilobase (kb) in both directions around the gene.

4. The system of claim 3, wherein the domain comprises more than 100 kb in both directions around the gene when the domain selector does not find an insulator within the 100 kb in both directions around the gene.

5. The system of claim 1, wherein the comparison is based on an expression level of the gene and a methylation level of the enhancer.

6. The system of claim 5, wherein the correlater determines that the enhancer pairs with the gene when the expression level is greater than a first threshold and the methylation level is greater than a second threshold.

7. The system if claim 6, wherein the first threshold or the second threshold is based on a Shannon Entropy Q value.

8. A method, comprising:
selecting, at a server comprising a non-transitory memory and a processor, a gene from a given cell type;
establishing, at the server, a domain across the gene;
identifying, at the server, an enhancer located within the domain; and
comparing, at the server, a pair of the enhancer and the gene to a known set of enhancer-gene pairs.

9. The method of claim 8, further comprising predicting, at the server, whether the enhancer is associated with the gene based on the comparison.

10. The method of claim 8, wherein the comparing further comprises:
determining an expression level of the gene;
determining a methylation level of the enhancer;
comparing the expression level of the gene to expression levels of the genes in the known set of enhancer-gene pairs; and
comparing the methylation level of the enhancer to methylation levels of the enhancers in the known set of enhancer-gene pairs.

11. The method of claim 10, wherein the comparing further comprises predicting that the enhancer pairs with the gene when the expression level is greater than a first threshold and the methylation level is greater than a second threshold.

12. The method of claim 11, wherein the first threshold or the second threshold is based on a Shannon Entropy Q value.

13. The method of claim 8, wherein the establishing the domain further comprises searching for an insulator within 100 kilobase (kb) in both directions around the gene.

14. The method of claim 13, further comprising defining the domain according to a location of an insulator in both directions around the gene.

15. The method of claim 14, wherein the domain is extended beyond 100 kb in one direction around the gene until the insulator is found or stopped short of 100 kb in one direction if an insulator is found.

16. A non-transitory computer readable storage medium storing machine-readable instructions that when executed by a processor facilitate the performance of operations, the operations comprising:
identifying a gene from a given cell type based on an expression level;
locating an enhancer within a domain established across the gene;
comparing the enhancer and the gene to a set of known enhancer-gene pairs; and
predicting whether the enhancer and the gene are associated based on the comparison.

17. The non-transitory computer readable storage medium of claim 16, wherein the operations further comprise:
setting a preliminary domain comprising at least 100 kilobase (kb) in both directions around the gene;
searching for an insulator in both directions around the gene;
setting the domain in both directions around the gene based on an identified inhibitor in both directions around the gene.

18. The non-transitory computer readable storage medium of claim 16, wherein the operations further comprise:
determining an expression level of the gene;
determining a methylation level of the enhancer;
comparing the expression level of the gene to expression levels of the genes in the known set of enhancer-gene pairs; and
comparing the methylation level of the enhancer to methylation levels of the enhancers in the known set of enhancer-gene pairs.

19. The non-transitory computer readable storage medium of claim 18, wherein the operations further comprise predicting that the enhancer pairs with the gene when the expression level is greater than a first threshold and the methylation level is greater than a second threshold.

20. The non-transitory computer readable storage medium of claim 19, wherein the first threshold or the second threshold is based on a Shannon Entropy Q value.
